(19) United States
(12) Patent Application Publication
(10) Pub. No.: US 2023/0303503 A1
(43) Pub. Date: Sep. 28, 2023

(54) CELL ACTIVATABLE IRON CHELATORS

(71) Applicant: BOARD OF REGENTS, THE UNIVERSITY OF TEXAS SYSTEM, Austin, TX (US)

(72) Inventors: Jonathan L SESSLER, Austin, TX (US); Adam C. SEDGWICK, Austin, TX (US); James Thomas BREWSTER, II, Austin, TX (US); Axel STEINBRÜCK, Austin, TX (US)

(73) Assignee: BOARD OF REGENTS, THE UNIVERSITY OF TEXAS SYSTEM, Austin, TX (US)

(21) Appl. No.: 17/906,913

(22) PCT Filed: Mar. 23, 2021
(86) PCT No.: PCT/US2021/023650

§ 371 Date: Sep. 21, 2022

Related U.S. Application Data
(60) Provisional application No. 63/000,047, filed on Mar. 26, 2020.
(60) Provisional application No. 62/993,689, filed on Mar. 23, 2020.

(51) Int. Cl. C07D 249/08; A61K 45/06; A61P 35/00; A61P 31/04; C07H 15/26; C07D 417/10; C07F 9/6558; A61K 49/00; C12Q 1/18

(52) U.S. Cl. CPC C07D 249/08; A61K 45/06; A61K 49/0021; A61P 31/04; A61P 35/00; C07D 417/10; C07F 9/65583; C07H 15/26; C12Q 1/18

(57) ABSTRACT

Provided herein are compounds of the formula (I): wherein the variables are as defined herein. Pharmaceutical compositions of the compounds are also provided. In some aspects, these compounds may be used for the treatment of diseases or disorders, such as a bacterial infection.

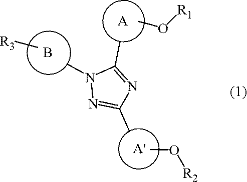

This application claims the benefit of U.S. Provisional Pat. Application No. 63/000,047, filed Mar. 26, 2020, and 62/993,689, filed Mar. 23, 2020, the entirety of both of which are incorporated herein by reference.

### BACKGROUND

### I. Field

The present disclosure relates generally to the fields of iron chelators. More particularly, it concerns compounds which act as masked iron chelators which are activated in the presence of a microorganism.

### II. Description of Related Art

Pandemics arising from a mircoorganisms are an emerging threat to the global population. These threats are particularly acute with the rapid evolution of bacteria. Bacterial evolution, coupled with the global misuse of antibiotic treatments, has led to the emergence of antibiotic resistant bacteria (so-called “superbugs”) towards which numerous antibiotics are inactive (Wright et al., 2014; von Bubnoff, 2006; Peeters et al., 2019). This is spawning serious public health con-cerns, including fears of a potential return to the pre-antibiotic era (Rugina, 2018). Cost effective strategies for overcoming antibiotic resistance and new agents that operate via novel mechanisms of action may help alleviate some of these concerns. Recently, researchers have begun to exploit siderophore-mediated iron up-take pathways using natural or synthetic Fe(III) chelators in an attempt to interfere with bacterial Fe(III) acquisition and augment host “nutritional immunity” antibacterial mechanisms (Negash, et al., 2019; Thompson et al., 2012; Miller and Malouin, 1993; Mislin and Schalk, 2014; Hennigar and McClung, 2016).

Similarly, such methods may be utilized to target a wide variety of microorganisms including parasites and viruses (WO 2016/203488).

Deferasirox (ExJade) is an FDA-approved treatment for iron overload disorders because of its ability to chelate Fe(II)/Fe(III) ions in vivo. While ExJade is FDA approved for treatment of these conditions, ExJade is associated with significant toxicity in patients and thus has limited usefulness. Therefore, there remains a desire to reduce this toxicity while maintaining its ability to chelate Fe(II)/Fe(III) ions for use as a treatment for an infection of a microorganism.

### SUMMARY OF THE INVENTION

The present disclosure provides compounds which may be used as Fe(II)/Fe(III) ion chelators which exhibit reduced toxicity.

In some aspects, the present disclosure provides compounds of the formula:

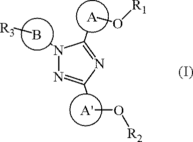

wherein:
A and A′ are arenediyl(_{c≤12}), heteroarenediyl(_{C≤12}), or a substituted version thereof;
B is —X₁—Y₁—, wherein:
   X₁ is a covalent bond, alkanediyl(_{C≤8}), substituted alkanediyl(_{C≤8}), arenediyl(_{c≤12}), or substituted arenediyl(_{C≤12}); and
   Y₁ is absent, -NR₄-, —C(O)—, —C(O)O—, -C(O)NR₄-, arenediyl(_{C≤12}), heteroarenediyl(_{C≤12}), heterocycloalkanediyl(_{C≤12}), or a substituted version wherein:
      R₄ is hydrogen, alkyl(_{C≤6)}, substituted alkyl(_{C≤6}), or a monovalent amine protecting group;
R₁ and R₂ are each independently a moiety cleavable to hydrogen; and
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
or a pharmaceutically acceptable salt thereof.

In some embodiments, the compounds are further defined as:

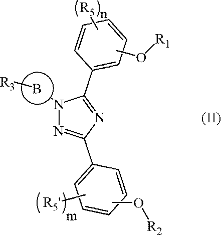

wherein:
B is —X₁—Y₂—, wherein:
   X₁ is a covalent bond, alkanediyl(_{C≤8}), substituted alkanediyl(_{C≤8}), arenediyl(_{c≤12}), or substituted arenediyl(_{C≤12}); and
   Y₁ is absent, -NR₄-, —C(O)—, —C(O)O—, -C(O)NR₄-, arenediyl(_{c≤12}), heteroarenediyl(_{C≤12}), heterocycloalkanediyl(_{C≤12}), or a substituted version wherein:
      R₄ is hydrogen, alkyl(_{C≤6)}, substituted alkyl(_{C≤6}), or a monovalent amine protecting group;
R₁ and R₂ are each independently a moiety cleavable to hydrogen;
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
R₅ and R₅′ are each independently hydrogen, halo, or hydroxy; and
m and n are each 1, 2, or 3;
or a pharmaceutically acceptable salt thereof.

In some embodiments, the compounds are further defined as:

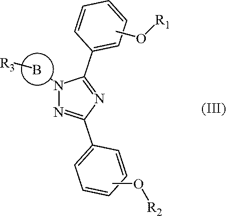

wherein:
B is —X₁—Y₁—, wherein:
   X₁ is a covalent bond, alkanediyl(_{C≤8}), substituted alkanediyl(_{C≤8}), arenediyl(_{c≤12}), or substituted arenediyl(_{C≤12}); and
   Y₁ is absent, -NR₄-, —C(O)—, —C(O)O—, -C(O)NR₄-, arenediyl(_{c≤12}), heteroarenediyl(_{C≤12}), heterocycloalkanediyl(_{C≤12}), heterocycloalkalkyl(_{C≤12}), or a substituted version wherein:
      R₄ is hydrogen, alkyl(_{C≤6)}, substituted alkyl(_{C≤6}), or a monovalent amine protecting group;
R₁ and R₂ are each independently a moiety cleavable to hydrogen; and
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
or a pharmaceutically acceptable salt thereof.

In some embodiments, the compounds are further defined as:

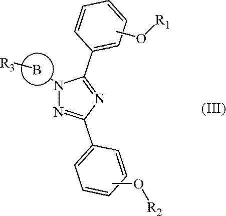

wherein:
B is —X₁—Y₁—, wherein:
   X₁ is arenediyl(_{C≤12}) or substituted arenediyl(_{C≤12}); and
   Y₁ is absent, —C(O)—, or —C(O)O—;
R₁ and R₂ are each independently a moiety cleavable to hydrogen; and
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
or a pharmaceutically acceptable salt thereof.

In some embodiments, A is arenediyl(_{C≤12}) or substituted arenediyl(_{C≤12}). In further embodiments, A is arenediyl(_{c≤12}), such as benzenediyl. In some embodiments, A′ is arenediyl(_{C≤12}) or substituted arenediyl(_{C≤12}). In further embodiments, A′ is arenediyl(_{C≤12}), such as benzenediyl. In some embodiments, R₅ is hydrogen. In other embodiments, R₅ is halo. In still other embodiments, R₅ is hydroxy. In some embodiments, R₅′ is hydrogen. In other embodiments, R₅′ is halo. In still other embodiments, R₅′ is hydroxy. In some embodiments, m is 1 or 2. In some embodiments, n is 1 or 2. In some embodiments, X₁ is a covalent bond. In other embodiments, X₁ is alkanediyl(_{C≤8}) or substituted alkanediyl(_{C≤8}). In further embodiments, X₁ is alkanediyl(_{C≤8}), such as methylene or ethylene. In still other embodiments, X₁ is arenediyl(_{C≤12}) or substituted arenediyl(_{C≤12}). In further embodiments, X₁ is arenediyl(_{c≤12}), such as benzenediyl. In some embodiments, Y₁ is absent. In other embodiments, Y₁ is —C(O)—. In still other embodiments, Y₁ is —C(O)O—. In other embodiments, Y₁ is -C(O)NR₄-. In some embodiments, R₄ is hydrogen. In other embodiments, R₄ is alkyl(_{C≤6)} or substituted alkyl(_{C≤6}). In further embodiments, R₄ is alkyl(_{C≤6}), such as methyl. In some embodiments, R₄ is substituted alkyl(_{C≤6)}, such as 2-hydroxyethyl.

In some embodiments, R₃ is hydrogen. In other embodiments, R₃ is alkyl(_{C≤12}) or substituted alkyl(_{C≤12}). In further embodiments, R₃ is alkyl(_{C≤12}), such as methyl or ethyl. In some embodiments, R₃ is substituted alkyl(_{C≤12}), such as 2-hydroxyethyl, 2-methoxyethyl, or 2,3-dihydroxyethyl. In other embodiments, R₃ is aryl(_{C≤12}) or substituted aryl(_{C≤12}). In further embodiments, R₃ is aryl(_{C≤12}), such as phenyl or napthyl. In some embodiments, R₃ is substituted aryl(_{C≤12}), such as 4-nitrophenyl, 4-methoxyphenyl, or 4-nitrophenyl. In still other embodiments, R₃ is aralkyl(_{C≤12}) or substituted aralkyl(_{C≤12}). In further embodiments, R₃ is aralkyl(_{C≤12}), such as benzyl. In other embodiments, R₃ is heteroaryl(_{C≤12}) or substituted heteroaryl(_{C≤12}). In further embodiments, R₃ is heteroaryl(_{C≤12}), such as pyridinyl or benzothiazolyl. In still other embodiments, R₃ is heterocycloalkyl(_{C≤12}) or substituted heterocycloalkyl(_{C≤12}). In further embodiments, R₃ is heterocycloalkyl(_{C≤12}), such as N-methylpiperazinyl, morpholinyl, or pyrrolidinyl. In yet other embodiments, R₃ is alkoxy(_{C≤12}) or substituted alkoxy(_{C≤12}). In further embodiments, R₃ is alkoxy(_{C≤12}), such as methoxy or ethoxy. In other embodiments, R₃ is heterocycloalkalkyl(c_{<12}) or substituted heterocycloalkalkyl(_{C≤12}). In further embodiments, R₃ is heterocycloalkalkyl(_{C≤12}), such as N′-methylpiperazinyl-N-2-ethyl. In still other embodiments, R₃ is alkylamino(_{C≤12}) or substituted alkylamino(_{C≤12}). In further embodiments, R₃ is alkylamino(_{C≤12}), such as methylamino or ethylamino. In some embodiments, R₃ is substituted alkylamino(_{C≤12}), such as 2-ethoxyethyl or 1-hydroxymethyl-2-hydroxyethyl. In yet other embodiments, R₃ is dialkylamino(_{C≤12}) or substituted dialkylamino(_{C≤12}). In further embodiments, R₃ is dialkylamino(_{C≤12}), such as dimethylamino or diethylamino. In some embodiments, R₃ is substituted dialkylamino(_{C≤12}), such as N,N-di-(2-hydroxyethyl)amino.

In some embodiments, R₁ or R₂ is moiety cleavable to hydrogen, wherein the moiety is cleaved by an enzyme that is substantially expressed by a microorganism. In some embodiments, the microorganism is a bacterium. In some embodiments, the enzyme is preferentially expressed by a microorganism. In some embodiments, the enzyme is exclusively expressed by a microorganism. In some embodiments, the enzyme is an esterase, a phosphatase, or an enzyme which cleaves a bond to a sugar group. In some embodiments, the enzyme is a phosphatase or an enzyme which cleaves a bond to a sugar group. In some embodiments, the enzyme which cleaves a bond to a sugar group is a glactosidase.

In some embodiments, R₁ or R₂ is a moiety cleavable to hydrogen, wherein the moiety is cleaved in response to inflammation. In some embodiments, the moiety is cleaved by a molecule generated as a part of the inflammation response. In some embodiments, the molecule is a reactive oxygen species, such as a superoxide or a peroxide. In other embodiments, the molecule is a chlorite, such as hypochlorite.

In some embodiments, R₁ or R₂ is a moiety cleavable to hydrogen, wherein the moiety is a therapeutic compound linked to the molecule by an ester, ether, hemiacetal, acetal, hemiketal, ketal, sulfonyl ether, sulfinyl ether, sulfonate ether, phosphate ester, boronate ester, or boronic acid. In some embodiments, the therapeutic compound is linked to the molecule by an ester group. In some embodiments, the therapeutic compound is aspirin.

In some embodiments, the moiety cleavable to hydrogen is further defined as a sugar or sugar derivative moiety or a functional group of the structure:

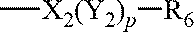

wherein:
X₂ is C, P, or S;
Y₂ is O or S;
p is 1 or 2; and
R₆ is hydrogen, amino, hydroxy, or alkyl(_{C≤12}), aryl(_{C≤12}), alkoxy(_{C≤12}), alkylamino(_{C≤12}), dialkylamino(_{C≤12}), or a substituted version thereof, or a therapeutic compound.

In some embodiments, X₂ is C. In other embodiments, X₂ is P. In still other embodiments, X₂ is S. In some embodiments, p is 1. In some embodiments, p is 2. In some embodiments, Y₂ is O. In other embodiments, Y₂ is S. In some embodiments, R₆ is dialkylamino(_{C≤12}) or substituted dialkylamino(_{C≤12}). In further embodiments, R₆ is dialkylamino(_{C≤12}), such as dimethylamino. In some embodiments, R₆ is alkyl(_{C≤12}) or substituted alkyl(_{C≤12}). In further embodiments, R₆ is alkyl(_{C≤12}), such as methyl. In some embodiments, R₆ is substituted alkyl(_{C≤12}), such as trifluoromethyl. In some embodiments, R₆ is aryl(_{C≤12}) or substituted aryl(_{C≤12}). In further embodiments, R₆ is substituted aryl(_{C≤12}), such as 2-acetoxyphenyl. In other embodiments, R₆ is hydroxy.

In some embodiments, the moiety cleavable to hydrogen is a sugar or sugar derivative moiety. In some embodiments, the sugar moiety is linked by the anomeric carbon atom. In some embodiments, the sugar moiety is a pentose or a hexose. In further embodiments, sugar moiety is fructose, glucose, or galactose. In some embodiments, R₁ and R₂ are the same group. In some embodiments, R₁ and R₂ are different groups.

In some embodiments, the compound is further defined as:

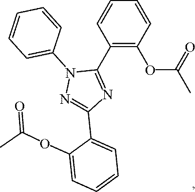

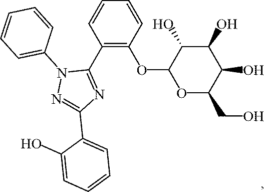

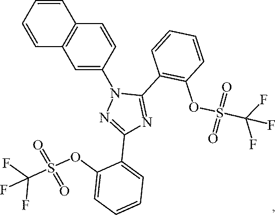

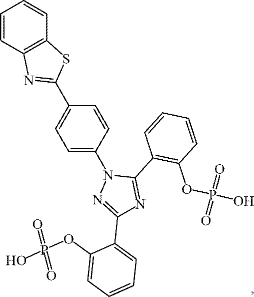

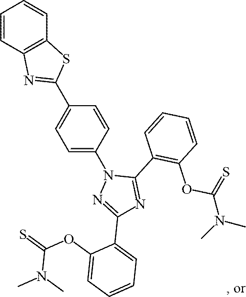

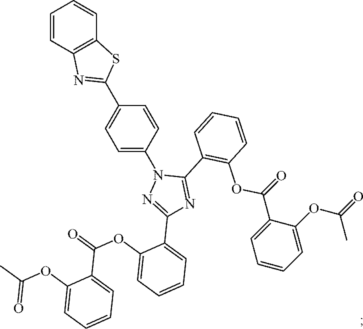

or a pharmaceutically acceptable salt thereof.

In other aspects, the present disclosure provides pharmaceutical compositions comprising: (A) a compound of the present disclosure; and (B) an excipient. In some embodiments, the composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In further embodiments, the composition is formulated for administration: topically, orally, or for injection. In some embodiments, the pharmaceutical composition is formulated as a unit dose.

In still yet another aspect, the present disclosure provides methods of treating a disease or disorder in a patient comprising administering to the patient in need thereof a therapeutically effective dose of a compound or pharmaceutical composition described herein. In some embodiments, the disease or disorder is an infection of a microorganism. In some embodiments, the microorganism is a bacterium. In other embodiments, the microorganism is a virus. In other embodiments, the disease or disorder is cancer. In some embodiments, the cancer is the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is lung cancer. In some embodiments, the methods further comprise administering a second therapeutic agent. In some embodiments, the second therapeutic agent is an antibiotic. In other embodiments, the second therapeutic agent is an anti-viral. In some embodiments, the patient is a mammal such as a human.

In another aspect, the present disclosure provides methods of imaging an infection comprising contacting the microorganism with a compound or composition described herein and detecting a change in signal. In some embodiments, the signal is a change in fluorescence. In some embodiments, the signal is change in the adsorbance of visible or untraviolet light. In some embodiments, the presence of the microorganism is indicated by the decrease in signal. In other embodiments, the presence of the microorganism is a change in the λₘₐₓ.

In some embodiments, the microorganism is imaged in vivo. In other embodiments, the microorganism is imaged in vitro. In other embodiments, the microorganism is imaged ex vivo. In some embodiments, the microorganism is a bacterium. In other embodiments, the microorganism is a virus. In other embodiments, the microorganism is a fungi. In some embodiments, the microorganism is present as a biofilm.

In still yet another aspect, the present disclosure provides methods of detecting a disease or disorder in a patient comprising contacting the patient with a compound or composition described herein, exposing the patient to a light source, and detecting a change in signal. In some embodiments, the signal is a change in fluorescence. In some embodiments, the signal is change in the adsorbance of visible or untraviolet light. In some embodiments, the disease or disorder is indicated by the decrease in signal. In other embodiments, the disease or disorder is a change in the λₘₐₓ. In some embodiments, the patient is imaged in vivo. In some embodiments, the disease or disorder is an infection of a microorganism. In some embodiments, the microorganism is a bacterium. In other embodiments, the microorganism is a virus. In other embodiments, the disease or disorder is cancer.

In some embodiments, the compound exhibits absorbance or fluorescence at two or more wavelengths. In some embodiments, the signal is a change in one of the two wavelengths. In some embodiments, the signal is a change at both of the wavelengths. In some embodiments, the methods further comprise administering a second compound or composition described herein. In some embodiments, the second compound or composition has a different signal. In some embodiments, the second compound or composition detects a different disease or disorder.

In still yet another aspect, the present disclosure provides methods of imaing a patient comprising administering to the patient a compound or composition described herein, exposing the patient to a light source, and measuring the resultant signal. In some embodiments, the signal is a change in fluorescence. In some embodiments, the signal is change in the adsorbance of visible or untraviolet light. In some embodiments, the signal decreases in intensity. In other embodiments, the signal is a change in the λₘₐₓ. In some embodiments, the patient is imaged in vivo. In some embodiments, the compound targets a microorganism or cellular structure. In some embodiments, the microorganism is a bacterium. In other embodiments, the microorganism is a virus. In some embodiments, the compound targets a cellular structure. In some embodiments, the compound exhibits absorbance or fluorescence at two or more wavelengths. In some embodiments, the signal is a change in one of the two wavelengths. In some embodiments, the signal is a change at both of the wavelengths. In some embodiments, the methods further comprise administering a second compound or composition described herein. In some embodiments, the second compound or composition has a different signal. In some embodiments, the second compound or composition targets a different microorganism or cellular structure.

In other aspect, the present disclosure provides compounds of the formula:

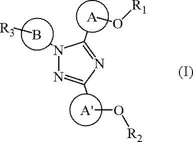

wherein:
A and A′ are arenediyl(_{c≤12}), heteroarenediyl(_{C≤12}), or a substituted version thereof;
B is —X₁—Y₁—, wherein:
   X₁ is arenediyl(_{C≤12}) or substituted arenediyl(_{C≤12}); and
   Y₁ is —C(O)—, —C(O)O—, or -C(O)NR₄-;
      R₄ is hydrogen, alkyl(_{C≤6)}, substituted alkyl(_{C≤6}), or a monovalent amine protecting group;
R₁ and R₂ are each indepdently hydrogen, alkyl(_{C≤12}), or substituted alkyl(_{C≤12}); and
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
provided that R₃ is not hydrogen, when Y₁ is —C(O)O—;
or a pharmaceutically acceptable salt thereof.

In some embodiments, the compounds are further defined as:

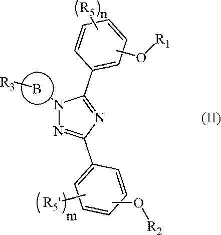

wherein:
B is —X₁—Y₁—, wherein:
   X₁ is arenediyl(_{C≤12}) or substituted arenediyl(_{C≤12}); and
   Y₁ is —C(O)—, —C(O)O—, or -C(O)NR₄-;
      R₄ is hydrogen, alkyl(_{C≤6)}, substituted alkyl(_{C≤6}), or a monovalent amine protecting group;
R₁ and R₂ are each indepdently hydrogen, alkyl(_{C≤12}), or substituted alkyl(_{C≤12}); and
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
R₅ and R₅′ are each independently hydrogen, halo, or hydroxy; and
m and n are each 1, 2, or 3;
or a pharmaceutically acceptable salt thereof.

In some embodiments, the compounds are further defined as:

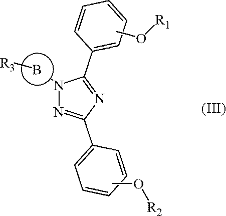

wherein:
B is ―X₁―Y₁―, wherein:
   X₁ is arenediyl(_{C≤12}) or substituted arenediyl(_{C≤12}); and
   Y₁ is —C(O)—, —C(O)O—, or -C(O)NR₄-;
      R₄ is hydrogen, alkyl(_{C≤6)}, substituted alkyl(_{C≤6}), or a monovalent amine protecting group;
R₁ and R₂ are each indepdently hydrogen, alkyl(_{C≤12}), or substituted alkyl(_{C≤12}); and
R₃ is hydrogen or alkyl(_{C≤12}), aryl(_{C≤12}), heteroaryl(_{C≤12}), heterocycloalkyl(_{C≤12}), aralkyl(_{C≤12}), heterocycloalkalkyl(_{C≤12}), alkoxy(_{C≤12}), alkylamino(_{C≤12}), dialkylamino(_{C≤12}), or a substituted version thereof;
or a pharmaceutically acceptable salt thereof.

In some embodiments, the compounds are further defined as:

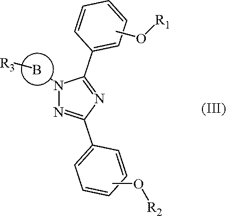

wherein:
B is —X₁—Y₁—, wherein:
   X₁ is arenediyl(_{C≤12}) or substituted arenediyl(_{C≤12}); and
   Y₁ is -C(O)NR₄-;
      R₄ is hydrogen, alkyl(_{C≤6)}, substituted alkyl(_{C≤6)}, or a monovalent amine protecting group;
R₁ and R₂ are each indepdently hydrogen, alkyl(_{C≤12}), or substituted alkyl(_{C≤12}); and
or a pharmaceutically acceptable salt thereof.

In some embodiments, A is arenediyl(_{C≤12}) or substituted arenediyl(_{C≤12}). In some embodiments, A is arenediyl(_{C≤12}) such as benzenediyl. In some embodiments, A′ is arenediyl(_{C≤12}) or substituted arenediyl(_{C≤12}). In some embodiments, A′ is arenediyl(_{C≤12}) such as benzenediyl. In some embodiments, X₁ is a covalent bond. In other embodiments, X₁ is arenediyl(_{C≤12}) such as benzenediyl.

In some embodiments, Y₁ is —C(O)—. In other embodiments, Y₁ is —C(O)O—. In other embodiments, Y₁ is -C(O)NR₄-. In some embodiments, R₄ is hydrogen. In other embodiments, R₄ is alkyl(_{C≤6)} or substituted alkyl(_{C≤6)}. In some embodiments, R₄ is alkyl(_{C≤6)} such as methyl.

In some embodiments, R₃ is hydrogen. In other embodiments, R₃ is alkyl(_{C≤12}) or substituted alkyl(_{C≤12}). In some embodiments, R₃ is alkyl(_{C≤12}) such as methyl or ethyl. In other embodiments, R₃ is substituted alkyl(_{C≤12}) such as 2-aminoethyl, 2-(dimethylamino)ethyl, 2-triphenylphosphiumethyl, or 2-cholylethyl. In other embodiments, R₃ is heterocycloalkyl(_{C≤12}) or substituted heterocycloalkyl(_{C≤12}). In some embodiments, R₃ is heterocycloalkyl(_{C≤12}) such as N-methylpiperazinyl or morpholinyl. In other embodiments, R₃ is alkoxy(_{C≤12}) or substituted alkoxy(_{C≤12}). In some embodiments, R₃ is alkoxy(_{C≤12}) such as methoxy. In other R₃ is alkylamino(_{C≤12}) or substituted alkylamino(_{C≤12}). In some embodiments, R₃ is alkylamino(_{C≤12}) such as methylamino. In other embodiments, R₃ is dialkylamino(_{C≤12}) or substituted dialkylamino(_{C≤12}). In some embodiments, R₃ is dialkylamino(_{C≤12}) such as dimethylamino.

In some embodiments, the compounds are further defind as:

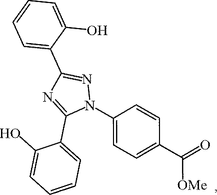

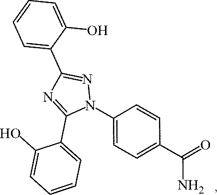

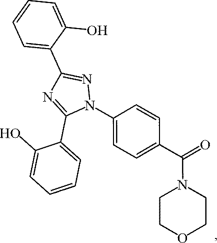

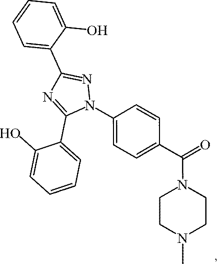

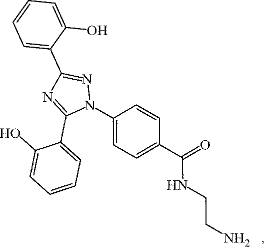

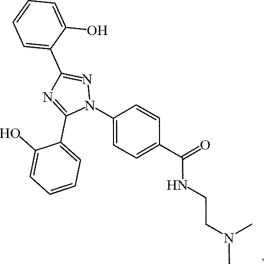

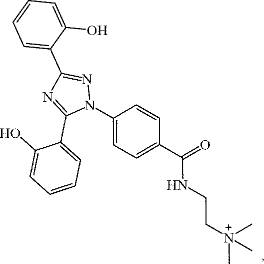

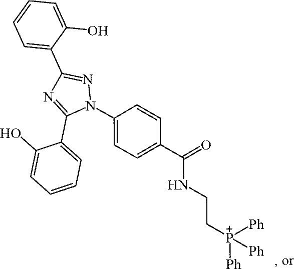

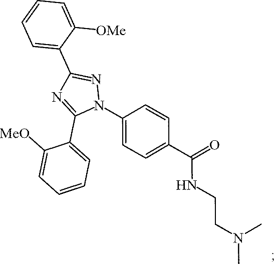

or a pharmaceutically acceptable salt thereof.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn’t mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description and examples provided herewith.

FIGS. 1A-1F shows the comparison of the toxicity of the compounds with cleavable groups on the phenolic hydoxides relative to the unmodified hydroxide groups. FIG. 1A shows ExPh_bisAcyl, FIG. 1B shows ExPh_monoGal, FIG. 1C shows ExNaph_bisOTf, FIG. 1D shows Ex_Phos, FIG. 1E shows ExBT_bisDTC, and FIG. 1F shows ExBT_bisAspirin.

FIGS. 2A-2D show (FIG. 2A) Ball and stick crystal structure of ExPh. (FIG. 2B) Ball and stick crystal structure of ExBT. (FIG. 2C) Fluorescence spectra of ExPh (10 µM) in THF and PBS (pH = 7.40); λₑₓ = 300 nm. (FIG. 2D) Fluorescence spectra of ExBT (10 µM) in THF and PBS (pH = 7.40); λₑₓ = 350 nm. CCDC 1967984 (ExPh); CCDC 1967981 (ExBT).

FIGS. 3A-3F show fluorescence spectra of (FIG. 3A) ExBT (15 µM), (FIG. 3B) ExBT-OMe, and (FIG. 3C) ExPhos (15 µM) recorded upon exposure to increasing Fe(III) concentrations (0-15 µM as the FeCl₃ salt). (FIG. 3D) Fluorescence changes of ExPhos (15 µM) with increasing alkaline phosphatase (ALP, 0-64 U). (FIG. 3E) Time-dependent change in the fluorescence-emission intensity at 505 nm observed when solutions containing ExPhos (15 µM) and ALP (two concentrations) were excited at 320 nm. (FIG. 3F) Fluorescence spectra of ExPhos (15 µM) preincubated with 64 U of ALP recorded as a function of increasing Fe(III) (0-15 µM). All measurements were carried out in deionized water.

FIGS. 4A-4C shows (FIG. 4A) MRSA (ATCC 43300) colonies on Luria-Bertani (LB) tryptone agar plates and VRE (ATCC 51299) colonies on Brain-Heart Infusion (BHI) tryptone agar plates before and after treatment with ExBT, ExJade, ExPhos, and arbekacin (15 µM). Cell viability of (FIG. 4B) MRSA (ATCC 43300) and (FIG. 4C) VRE (ATCC 51299) before and after treatment with ExBT, ExJade, ExPhos, and arbekacin (15 µM).

FIGS. 5A & 5B shows (FIG. 5A) Confocal Laser-Scanning Microscope (CLSM) images (left: fluorescence images; right: bright-field images) of MRSA (ATCC 43300) treated with ExBT (40 µM), ExPhos (40 µM), or ExPhos pretreated with different concentrations of a phosphatase inhibitor cocktail A (+ 50 µM sodium fluoride, 10 µM sodium pyrophosphate, 10 µM β-glycerophosphate, and 10 µM sodium orthovanadate; ++: 100 µM sodium fluoride, 20 µM sodium pyrophosphate, 20 µM β-glycerophosphate and 20 µM sodium orthovanadate). (FIG. 5B) Normalized fluorescence intensities of the fluorescent imaged systems. ***P<0.001.

FIGS. 6A & 6B show (FIG. 6A) chemical structures of deferasirox (ExJade) and the derivatives used in this study. (FIG. 6B) Basic schematic of the alkaline phosphatase (ALP)-mediated hydrolysis of exphos to AIE-active ExBT, followed by its fluorescence quenching mediated by Fe(III) chelation.

FIGS. 7A-7D show fluorescence spectra of (FIG. 7A) ExPh and (FIG. 7B) ExBT (10 µM) in MeCN and H₂O; λₑₓ = 310 and 350 nm, respectively (slit widths ex = 5 nm, em = 5 nm). (FIG. 7C) Change in normalized emission intensity of ExPh (10 µM) at 480 nm with increasing water fractions (ƒ_{w}, H₂O/MeCN) (λₑₓ = 310 nm, slit widths ex = 5 nm, em = 5 nm). (FIG. 7D) Change in normalized emission intensity of ExBT (10 µM) at 530 nm with increasing water fractions (fw, H₂O/MeCN) (λₑₓ = 350 nm, slit widths ex = 5 nm, em = 5 nm). Relative quantum yields (Φ_{F}) of ExPh and ExBT in water (reference: quinine sulfate, 0.1 M H₂SO₄) were determined as 0.082 and 0.13, respectively (MeCN: ExPh Φ_{F} = -0.0011, ExBT Φ_{F} = 0.0037).

FIGS. 8A-8D show (FIG. 8A) ball and stick view of the X-ray diffraction structure of ExPh. (FIGS. 8B-8D) Molecular stacking of ExPh shown in different views. CCDC no. 1967984 ExPh.

FIGS. 9A & 9B show top and side views of geometry-optimized structures of ExPh and the corresponding natural transition orbitals (NTOs). (FIG. 9A) Ground state and (FIG. 9B) excited state relaxed geometries.

FIGS. 10A-10F show fluorescence spectra of (FIG. 10A) ExBT (15 µM), (FIG. 10B) ExBT-OMe (15 µM), and (FIG. 10C) ExPhos (15 µM) recorded upon exposure to Fe(III) (0-300 µM as the FeCl₃ salt). (FIG. 10D) Fluorescence spectra of ExPhos (15 µM) preincubated with 64 U of ALP followed by exposure to Fe(III) (0-300 µM). (FIG. 10E) Fluorescence changes of ExPhos (15 µM) with increasing alkaline phosphatase (ALP, 0-64 U). (FIG. 10F) Time-dependent change in the fluorescence-emission intensity (505 nm) of ExPhos (15 µM) when exposed to ALP (two concentrations, 32 U and 64 U). All measurements were carried out in deionized water and excited at 320 nm.

FIGS. 11A & 11B show biofilm experiments. (FIG. 11A) Evaluation of CFP-SUL (cefoperazone-sulbactam; 16 µg/mL), CFP-SUL@ExBT (16 µg/mL@64 µg/mL), CFP-SUL@deferasirox (16 µg/mL@64 µg/mL), and CFP-SUL@ExPhos (16 µg/mL@64 µg/mL) for the treatment of P. aeruginosa and MRSA biofilms. Processed 3D images of P. aeruginosa and MRSA biofilms on glass slides using the live/dead biofilm viability assay. Distributions of PI-stained (red, dead bacteria) and Syto9-stained (green, live bacteria) (FIG. 11B) P. aeruginosa and MRSA biovolumes and CFU counts (purple dots) for the above groups. Biovolume data are mean values of three independent replicates.

FIGS. 12A & 12B show (FIG. 12A) Confocal laser-scanning microscope (CLSM) images of ExBT (15 µM) and ExPhos (15 µM) associated with treatment of P. aeruginosa and MRSA biofilms at different times. (FIG. 12B) Normalized intensities of the fluorescent images.

FIGS. 13A & 13B show (FIG. 13A) Structures of 1 and its complex with Fe³⁺ (Steinhauser et al., 2004) (FIG. 13B) Conditions: (i) Methanol, reflux, 48 h. (ii) Urea (4 equiv.), imidazole (2 equiv.), microwave 150 W, 170° C., 20 min. (iii) EDC (2 equiv.), TEA (2 equiv.), NHS (cat.), amine (3 equiv.), CH₂Cl₂, r.t., 16 h. *Isolated after anion exchange with aqueous NaPF₆. (iv) Same as (iii) with N-BOC ethylenediamine, then TFA, r.t., 16 h.

FIG. 14 shows selected proliferation profiles of 1, 8, 10 and oxaliplatin (Ox-Pt) used as a positive control. The high HS parameter of 8 translates to sharp decline in cell viability with increasing drug concentrations. Derivative 8 also achieves baseline eradication of cancer cells at the lowest concentration of all evaluated drugs.

FIGS. 15A & 15B shows (FIG. 15A) normalized emission spectra of 2 and 8, respectively, recorded at 50 µM in PBS with excitation at 360 nm. (FIG. 15B) Confocal microscopy imaging of 2 and 8 (20 µM). Colocalization was observed between 8 and Lysotracker® red. Blue channel: Ex/Em = 405/ 440-480 nm. Red channel: Ex/Em = 559/ 580-620 nm.

### DESCRIPTION OF ILLUSTRATIVE EMBODIMENTS

The compounds described herein are derivatives of ExJade which contain reactive groups on the phenol chelating groups which are cleavable in the presence of one or more types of external stimuli. These compounds may be responsive to stimuli such as inflammation markers such as perchlorite or reactive oxygen species such as superoxides and peroxides or be cleaved by a cellular enzyme. In some embodiments, the cellular enzyme is one such as a phosphatase or sugar cleaving enzyme that are predominately present in the cells of a microorganism such as a virus, fungus, protozoan, or bacteria. In some embodiments, these enzymes are specifically expressed by the microorganism. The compounds described herein may be used in therapeutic applications with reduced overall toxicity to normal human tissues than the compounds wherein the phenol group has not yet been modified with a cleavable group.

### I. Derivatives of ExJade

The ExJade derivatives described herein (also described as derivatives of ExJade) are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March’s Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development - A Guide for Organic Chemists (2012), which is incorporated by reference herein.

All the ExJade derivatives described herein may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the ExJade derivatives described herein are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the ExJade derivatives described herein have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

ExJade derivatives described herein may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the ExJade derivatives described herein can have the S or the R configuration. In some embodiments, the present compounds may contain two or more atoms which have a defined stereochemical orientation.

Chemical formulas used to represent ExJade derivatives described herein will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the ExJade derivatives described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, ExJade derivatives described herein function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclsoure contemplates that the ExJade derivatives described herein may function as prodrugs as well as methods of delivering prodrugs. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, ExJade derivatives described herein exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### Ii. Infections of a Microorganism

In some aspects of the present disclosure, the ExJade derivatives described herein may be used to treat a microbial infection (an infection of a microorganism). Some non-limiting examples of mircoorganisms which may be treated with the compounds herein include bacteria, viruses, parasites, and fungi.

### 1. Baterial Infections

In some aspects, the present disclosure provides ExJade derivatives described hrein that may be used to treat a bacterial infection. While humans contain numerous different bacteria on and inside their bodies, an imbalance in bacterial levels or the introduction of pathogenic bacteria can cause a symptomatic bacterial infection. Pathogenic bacteria cause a variety of different diseases including but not limited to numerous foodborne illness, typhoid fever, tuberculosis, pneumonia, syphilis, and leprosy.

Additionally, different bacteria have a wide range of interactions with body and those interactions can modulate ability of the bacteria to cause an infection. For example, bacteria can be conditionally pathogenic such that they only cause an infection under specific conditions. For example, Staphylococcus and Streptococcus bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacteria can also be intracellular pathogens which can grow and reproduce within the cells of the host organism. Such bacteria can be divided into two major categories as either obligate intracellular parasites or facultative intracellular parasites. Obligate intracellular parasites require the host cell in order to reproduce and include such bacteria as but are not limited to Chlamydophila, Rickettsia, and Ehrlichia which are known to cause pneumonia, urinary tract infections, typhus, and Rocky Mountain spotted fever. Facultative intracellular parasites can reproduce either intracellular or extracellular. Some non-limiting examples of facultative intracellular parasites include Salmonella, Listeria, Legionella, Mycobacterium, and Brucella which are known to cause food poisoning, typhoid fever, sepsis, meningitis, Legionnaire’s disease, tuberculosis, leprosy, and brucellosis.

The ExJade derivatives described herein may be used in the treatment of bacterial infections, including those caused by Staphyloccoccus aureus. S. aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains.

Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). The methods described herein may be used in the treatment of MRSA bacterial strains.

Additionally, the ExJade derivatives described herein may be used to treat a Steptococcus pneumoniae infection. Streptococcus pneumoniae is a gram-positive, alpha-hemolytic, bile soluble aerotolerant anaerobe and a member of the genus Streptococcus. A significant human pathogenic bacterium, S. pneumoniae was recognized as a major cause of pneumonia in the late 19th century and is the subject of many humoral immunity studies.

Despite the name, the organism causes many types of pneumococcal infection other than pneumonia, including acute sinusitis, otitis media, meningitis, bacteremia, sepsis, osteomyelitis, septic arthritis, endocarditis, peritonitis, pericarditis, cellulitis, and brain abscess. S. pneumoniae is the most common cause of bacterial meningitis in adults and children, and is one of the top two isolates found in ear infection, otitis media. Pneumococcal pneumonia is more common in the very young and the very old.

S. pneumoniae can be differentiated from S. viridans, some of which are also alpha hemolytic, using an optochin test, as S. pneumoniae is optochin sensitive. S. pneumoniae can also be distinguished based on its sensitivity to lysis by bile. The encapsulated, gram-positive coccoid bacteria have a distinctive morphology on gram stain, the so-called, “lancet shape.” It has a polysaccharide capsule that acts as a virulence factor for the organism; more than 90 different serotypes are known, and these types differ in virulence, prevalence, and extent of drug resistance.

S. pneumoniae is part of the normal upper respiratory tract flora but as with many natural flora, it can become pathogenic under the right conditions (e.g., if the immune system of the host is suppressed). Invasins such as Pneumolysin, an anti-phagocytic capsule, various adhesins and immunogenic cell wall components are all major virulence factors.

Finally, bacterial infections could be targeted to a specific location in or on the body. For example, bacteria could be harmless if only exposed to the specific organs, but when it comes in contact with a specific organ or tissue, the bacteria can begin replicating and cause a bacterial infection.

### A. Gram-Positive Bacteria

In some aspects of the present disclosure, the ExJade derivatives described herein may be used to treat a bacterial infection by a gram-positive bacteria. Gram-positive bacteria contain a thick peptidoglycan layer within the cell wall which prevents the bacteria from releasing the stain when dyed with crystal violet. Without being bound by theory, the gram-positive bacteria are often more susceptible to antibiotics. Generally, gram-positive bacteria, in addition to the thick peptidoglycan layer, also comprise a lipid monolayer and contain teichoic acids which react with lipids to form lipoteichoic acids that can act as a chelating agent. Additionally, in gram-positive bacteria, the peptidoglycan layer is outer surface of the bacteria. Many gram-positive bacteria have been known to cause disease including, but are not limited to, Streptococcus, Straphylococcus, Corynebacterium, Enterococcus, Listeria, Bacillus, Clostridium, Rathybacter, Leifsonia, and Clavibacter.

### B. Gram-Negative Bacteria

In some aspects of the present disclosure, the ExJade derivatives described herein may be used to treat a bacterial infection by a gram-negative bacteria. Gram-negative bacteria do not retain the crystal violet stain after washing with alcohol. Gram-negative bacteria, on the other hand, have a thin peptidoglycan layer with an outer membrane of lipopolysaccharides and phospholipids as well as a space between the peptidoglycan and the outer cell membrane called the periplasmic space. Gram-negative bacterial generally do not have teichoic acids or lipoteichoic acids in their outer coating. Generally, gram-negative bacteria also release some endotoxin and contain prions which act as molecular transport units for specific compounds. Most bacteria are gram-negative. Some non-limiting examples of gram-negative bacteria include Bordetella, Borrelia, Burcelia, Campylobacteria, Escherichia, Francisella, Haemophilus, Helicobacter, Legionella, Leptospira, Neisseria, Pseudomonas, Rickettsia, Salmonella, Shigella, Treponema, Vibrio, and Yersinia.

### C. Gram-Indeterminate Bacteria

In some aspects of the present disclosure, the ExJade derivatives described herein may be used to treat a bacterial infection by a gram-indeterminate bacteria. Gram-indeterminate bacteria do not full stain or partially stain when exposed to crystal violet. Without being bound by theory, a gram-indeteriminate bacteria may exhibit some of the properties of the gram-positive and gram-negative bacteria. A non-limiting example of a gram-indeterminate bacteria include Mycobacterium tuberculosis or Mycobacterium leprae.

### 2. Viral Infections

In some aspects of the present disclosure, the compounds disclosed herein may be used to treat a viral infection. Similarly, virus can also exist in pathogenic form which can lead to human diseases. Viral infections are typically not treated directly but rather symptomatically since virus often have a self-limiting life cycle. Viral infections can also be more difficult to diagnosis than a bacterial infection since viral infections often do result in the concomitant increase in white blood cell counts. Some non-limiting examples of pathogenic virus include influenza virus, coronavirus, smallpox, BK virus, JC virus, human papillomavirus, adenovirus, herpes simplex type 1, herpes simplex type 2, varicella-zoster virus, Epstein barr virus, human cytomegalovirus, human herpesvirus type 8, Norwalk virus, human bocavirus, rubella virus, hepatitis E virus, hepatitis B virus, human immunodeficiency virus (HIV), Ebola virus, rabies virus, rotavirus, and hepatitis D virus.

### III. Hyperproliferative Diseases

While hyperproliferative diseases can be associated with any medical disorder that causes a cell to begin to reproduce uncontrollably, the prototypical example is cancer. One of the key elements of cancer is that the normal apoptotic cycle of the cell is interrupted and thus agents that lead to apoptosis of the cell are important therapeutic agents for treating these diseases. As such, the ExJade derivatives described herein may be effective in treating cancers.

Cancer cells that may be treated with the compounds according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget’s disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi’s sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing’s sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin’s disease; Hodgkin’s; paragranuloma; malignant lymphoma, small lymphocytic; malignant lymphoma, large cell, diffuse; malignant lymphoma, follicular; mycosis fungoides; other specified non-Hodgkin’s lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### IV. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of an ExJade derivative described herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient’s diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form (also known as a unit dose) for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$\begin{matrix}\text{HED} \left(\text{mg} / \text{kg}\right) = \\ \text{Animal} \text{dose} \left(\text{mg} / \text{kg}\right) \times \left(\text{Animal} {\text{K}}_{\text{m}} / \text{Human} {\text{K}}_{\text{m}}\right)\end{matrix}$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of an ExJade derivative or composition described herein comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### V. Combination Therapy

In addition to being used as a monotherapy, the compounds of the present disclosure may also be used in combination therapies with an additional antimicrobial agent such as an antibiotic or a compound which mitigates one or more of the side effects experienced by the patient.

These therapies would be provided in a combined amount effective to achieve a reduction in one or more disease parameter(s). This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the ExJade derivatives described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations. Other potential combinations will be apparent to the skilled practitioner.

It also is conceivable that more than one administration of either the compound or the other therapy will be desired. Various combinations may be employed, where a compound of the present disclosure is “A,” and the other therapy is “B,” as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B
Agents or factors suitable for use in a combined therapy with agents according to the present disclosure against an infectious disease include antibiotics such as penicillins, cephalosporins, carbapenems, macrolides, aminoglycosides, quinolones (including fluoroquinolones), sulfonamides and tetracylcines. Other combinations are contemplated.

### 1. Antibiotics

The term “antibiotics” are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

The first commericallly available antibiotic was released in the 1930′s. Since then, many different antibiotics have been developed and widely prescribed. In 2010, on average, 4 in 5 Americans are prescribed antibiotics annually. Given the prevalence of antibiotics, bacteria have started to develop resistance to specific antibiotics and antibiotic mechanisms. Without being bound by theory, the use of antibiotics in combination with another antibiotic may modulate resistance and enhance the efficacy of one or both agents.

In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

### 2. Anti-viral

The term “antiviral” or “antiviral agents” are drugs which may be used to treat a viral infection. In general, antiviral agents act via two major mechanisms: preventing viral entry into the cell and inhibiting viral synthesis. Without being bound by theory, viral replication can be inhibited by using agents that mimic either the virus-associated proteins and thus block the cellular receptors or using agents that mimic the cellular receptors and thus block the virus-associated proteins. Furthermore, agents which cause an uncoating of the virus can also be used as antiviral agents.

The second mechanism of viral inhibition is preventing or interrupting viral synthesis. Such drugs can target different proteins associated with the replication of viral DNA including reverse transcriptase, integrase, transcription factors, or ribozymes. Additionally, the therapeutic agent interrupts translation by acting as an antisense DNA strain, inhibiting the formation of protein processing or assembly, or acting as virus protease inhibitors. Finally, an anti-viral agent could additionally inhibit the release of the virus after viral production in the cell.

Additionally, anti-viral agents could modulate the bodies own immune system to fight a viral infection. Without being bound by theory, the anti-viral agent which stimulates the immune system may be used with a wide variety of viral infections.

In some embodiments, the present disclosure provides methods of using the disclosed ExJade derivatives in a combination therapy with an anti-viral agent as described above. In some non-limiting examples, the anti-viral agent is abacavir, aciclovir, acyclovir, adefovir, amantadine, amprenavir, ampligen, arbidol, atazanavir, atripla, balavir, boceprevirertet, cidofovir, combivir, dolutegravir, daruavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, interferon type I, type II, and type III, lamivudine, lopinavir, loviride, maraviroc, moroxydine, methisazone, nelfinavir, nevirapine, nexavir, oseltamivir, penciclovir, peramivir, pleconaril, podophyllotoxin, raltegravir, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, traporved, valaciclovir, valganciclovir, vicriviroc, vidarabine, viramidine, zalcitabine, zanamivir, or zidovudine. In some embodiments, the anti-viral agents is an anti-retroviral, a fusion inhibitor, an integrase inhibitor, an interferon, a nucleoside analogues, a protease inhibitor, a reverse transcriptase inhibitor, a synergistic enhancer, or a natural product such as tea tree oil.

### 3. Antiparasitic Agents

In some aspects, the present disclosure provides ExJade derivatives described herein which may be used in combination with one or more antiparasitic agents selected from quinine, chloroquine, amodiaquine, proguanil, cycloquanil, sulfadoxine, primaquine, pyrimethamine, chlorproquanil, tetracycline, dapsone, doxycycline, clindamycin, mefloquine, halofantrine, bulaquine, artemisinin, artemether, arteether, atovaquone, lumefantrine, dihydroartemisinin, piperaquine, artesunate, pyronaridine, azithromycin, tafenoquine, trimethoprim, sulfamethoxazole, artemisone, ferroquine, fosmidomycin, tinidazole, naphthoquine, methylene blue, (+)-erythromefloquine, tert-butyl isoquine, trioxaquine, an endoperoxide, a dihydrofolate reductase inhibitor, or a dihydroorotate dehydrogenase inhibitor.

### 4. Other Therapies

In some embodiments, the ExJade derivatives described herein may be used in combination with hydrogen peroxide or any other peroxide compounds such as an organic peroxide. Additionally, the compounds may be administered with a compound or enzyme which is known to generate peroxide. Without wishing to be bound by any theory, it is believed that the hydrogen peroxide may assist in chelation of the compound with Fe(II)/Fe(III) or it may be itself be useful in the underlying therapeutic application.

Additionally, the ExJade derivatives described herein may be used in combination with a photosensitizer. Furthermore, it is contemplated that the compounds of the present disclosure can be used in combination with photodynamic therapy. Dosages of about 1.0 or 2.0 mg/kg to about 4.0 or 5.0 mg/kg, preferably 3.0 mg/kg may be employed, up to a maximum tolerated dose that was determined in one study to be 5.2 mg/kg. The photosensitizer may be administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

The co-administration of a sedative (e.g., benzodiazapenes) and narcotic analgesic are sometimes recommended prior to light treatment along with topical administration of Emla cream (lidocaine, 2.5% and prilocaine, 2.5%) under an occlusive dressing. Other intradermal, subcutaneous and topical anesthetics may also be employed as necessary to reduce discomfort. Subsequent treatments can be provided after approximately 21 days. The treating physician may choose to be particularly cautious in certain circumstances and advise that certain patients avoid bright light for about one week following treatment.

In one non-limiting example, when employing photodynamic therapy, a target area is treated with light at the full width half max delivered by LED device or an equivalent light source (e.g., a Quantum Device Qbeam™ Q BMEDXM-728 Solid State Lighting System, which operates at 728 nm) at an intensity of 75 mW/cm² for a total light dose of 150 J/cm². The light treatment takes approximately 33 minutes.

The optimum length of time following administration of the photosensitizing compound until light treatment can vary depending on the mode of administration, the form of administration, and the type of target tissue. Typically, the photosensitizing compound may persist for a period of minutes to hours, depending on the compound, the formulation, the dose, the infusion rate, as well as the type of tissue and tissue size.

After the photosensitizing compound has been administered, the tissue being treated is photoirradiated at a wavelength similar to the absorbance of the photosensitizing compound, such wavelengths may be either about 400-500 nm or about 700-800 nm, more preferably about 450-500 nm or about 710-760 nm, or most preferably about 450-500 nm or about 725-740 nm. The light source may be a laser, a light-emitting diode, or filtered light from, for example, a xenon lamp; and the light may be administered topically, endoscopically, or interstitially (via, e.g., a fiber optic probe). Preferably, the light is administered using a slit-lamp delivery system. The fluence and irradiance during the photoirradiating treatment can vary depending on type of tissue, depth of target tissue, and the amount of overlying fluid or blood. For example, a total light energy of about 100 J/cm² can be delivered at a power of 200 mW to 250 mW depending upon the target tissue.

### VI. Definitions

The definitions below supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### A. Chemical Groups

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; “boronic acid” means —B(OH)₂; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; “sulfonate” means —OS(O)₂—; and “sulfinyl” means —S(O)—.

In the context of chemical formulas, the symbol “-” means a single bond, “=” means a double bond, and “≡” means triple bond. The symbol

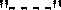

represents an optional bond, which if present is either single or double. The symbol

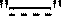

represents a single bond or a double bond. Thus, the formula

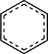

covers, for example,

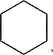

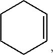

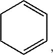

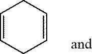

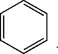

And it is understood that no one such ring atom forms part of more than one double bond. Furthermore, it is noted that the covalent bond symbol

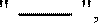

when connecting one or two stereogenic atoms, does not indicate any preferred stereochemistry. Instead, it covers all stereoisomers as well as mixtures thereof. The symbol

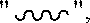

when drawn perpendicularly across a bond (e.g.,

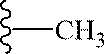

for methyl) indicates a point of attachment of the group. It is noted that the point of attachment is typically only identified in this manner for larger groups in order to assist the reader in unambiguously identifying a point of attachment. The symbol

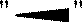

means a single bond where the group attached to the thick end of the wedge is “out of the page.” The symbol

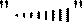

means a single bond where the group attached to the thick end of the wedge is “into the page”. The symbol

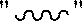

means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

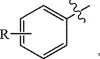

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

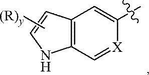

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl(_{C≤8})”, “alkanediyl(_{C≤8})”, “heteroaryl(_{C≤8})”, and “acyl(_{C≤8})” is one, the minimum number of carbon atoms in the groups “alkenyl(_{C≤8})”, “alkynyl(_{C≤8})”, and “heterocycloalkyl(_{C≤8})” is two, the minimum number of carbon atoms in the group “cycloalkyl(_{C≤8})” is three, and the minimum number of carbon atoms in the groups “aryl(_{C≤8})” and “arenediyl(_{C≤8})” is six. “Cn-n′” defines both the minimum (n) and maximum number (n′) of carbon atoms in the group. Thus, “alkyl(_{C2-10})” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl(_{C1-4})”, and “alkyl(_{C≤4})” are all synonymous. Except as noted below, every carbon atom is counted to determine whether the group or compound falls with the specified number of carbon atoms. For example, the group dihexylamino is an example of a dialkylamino(_{C12}) group; however, it is not an example of a dialkylamino_{(C6)} group. Likewise, phenylethyl is an example of an aralkyl(_{C=8}) group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n +2 electrons in a fully conjugated cyclic π system. An aromatic compound or chemical group may be depicted as a single resonance structure; however, depiction of one resonance structure is taken to also refer to any other resonance structure. For example:

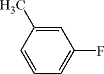

is also taken to refer to

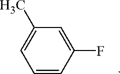

Aromatic compounds may also be depicted using a circle to represent the delocalized nature of the electrons in the fully conjugated cyclic π system, two non-limiting examples of which are shown below:

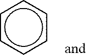

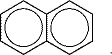

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neopentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group =CRR’ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

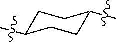

is a non-limiting example of cycloalkanediyl group. A “cycloalkane” refers to the class of compounds having the formula H-R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H-R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

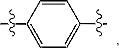

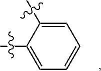

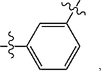

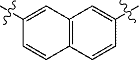

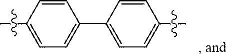

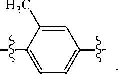

An “arene” refers to the class of compounds having the formula H-R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

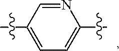

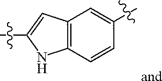

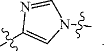

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

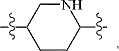

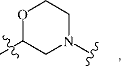

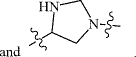

The term “heteroaralkyl” refers to the monovalent group -alkanediyl-heterocycloalkyl, in which the terms alkanediyl and heterocycloalkyl are each used in a manner consistent with the definitions provided above. Non-limiting examples are: 2-morpholinoethyl and piperindyl-methyl.

The term “acyl” refers to the group -C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group -C(O)R has been replaced with a sulfur atom, -C(S)R. The term “aldehyde” corresponds to an alkyl group, as defined above, attached to a —CHO group.

The term “acetal” is used to describe a carbonyl group which have reacted with two hydroxy or a dihydroxy containing compounds to form a diether of a germinal diol of the structure R₂C(OR′)₂ arising from the carbonyl group of the structure: R₂C(O), wherein neither R′ is not hydrogen and each R′ may be the same, different, or may be taken together to form a ring. A “mixed acetal” is an acetal wherein R′ are both different. “Acetal” may be used to describe the carbonyl group, which is an aldehyde, wherein one or both R groups are hydrogen atoms, or a ketone, wherein neither R group is a hydrogen atom. “Ketal” is a subgroup of “acetal” wherein the carbonyl group is a ketone. The term “hemiacetal” is used to describe a carbonyl group which has been reacted with one hydroxy containing compound to form a monoether of a germinal diol forming a group of the structure: R₂C(OH)OR′, wherein R′ is not hydrogen. “Hemiacetal” may be used to describe the carbonyl group that is an aldehyde, wherein one or both R groups are hydrogen atoms, or a ketone, wherein neither R group is a hydrogen atom. Analogous to “ketal”, a “hemiketal” is a subgroup of “hemiacetal” wherein the carbonyl group is a ketone.

The term “alkoxy” refers to the group -OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as -OR, in which R is cycloalkyl, alkenyl, alkynyl, aryl, aralkyl, heteroaryl, heterocycloalkyl, and acyl, respectively. The term “alkylthio” and “acylthio” refers to the group -SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group -NHR, in which R is an alkyl, as that term is defined above. Non-limiting examples include: —NHCH₃ and —NHCH₂CH₃. The term “dialkylamino” refers to the group -NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as -NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as -NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as -NRR′, in which R is alkyl and R′ is cycloalkyl. The term “amido” (acylamino), when used without the “substituted” modifier, refers to the group -NHR, in which R is acyl, as that term is defined above. A non-limiting example of an amido group is —NHC(O)CH₃.

The terms “alkylsulfonyl” and “alkylsulfinyl” refers to the groups -S(O)₂R and -S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner.

The term “alkylphosphate” refers to the group -OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” refers to the group -OP(O)(OR)(OR′), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. In other embodiments, the chemical group may also be substituted with a charged moiety like: P(R)₃⁺ or N(R)₃⁺, wherein R is an alkyl_{(C≤12)}, aryl_{(C≤12)}, or a substituted version of either group, wherein the substituted group is not a charged moiety. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

An “amine protecting group” or “amino protecting group” is well understood in the art. An amine protecting group is a group which modulates the reactivity of the amine group during a reaction which modifies some other portion of the molecule. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; alkylaminocarbonyl groups (which form ureas with the protect amine) such as ethylaminocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH- or PG_{DA}N- wherein PG_{MA} is a monovalent amine protecting group, which may also be described as a “monovalently protected amino group” and PG_{DA} is a divalent amine protecting group as described above, which may also be described as a “divalently protected amino group”.

A “moiety cleavable to hydrogen” means any group that is convertible to a hydrogen atom by enzymological or chemical means including, but not limited to, hydrolysis and hydrogenolysis. Non-limiting examples include hydrolyzable groups, such as acyl groups, groups having an oxycarbonyl group, amino acid residues, peptide residues, o-nitrophenylsulfenyl, trimethylsilyl, tetrahydropyranyl, and diphenylphosphinyl. Non-limiting examples of acyl groups include formyl, acetyl, and trifluoroacetyl. Non-limiting examples of groups having an oxycarbonyl group include ethoxycarbonyl, tert-butoxycarbonyl (-C(O)OC(CH₃)₃), benzyloxycarbonyl, p-methoxybenzyloxycarbonyl, vinyloxycarbonyl, and β-(p-toluenesulfonyl)ethoxycarbonyl. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Non-limiting examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), and tert-butoxycarbonyl groups (—C(O)OC(CH₃)₃). Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Non-limiting examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), and tert-butoxycarbonyl groups (—C(O)OC(CH₃)₃). Other examples of substituents “moiety cleavable to hydrogen” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl). In some embodiments, the functional group may have a structure:

### B. Other Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Absent one of the of the above measurements, the term “about” means ±5%.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤ 15%, more preferably ≤ 10%, even more preferably ≤ 5%, or most preferably ≤ 1% of another stereoisomer(s).

As used herein, the term “substantially expressed” means that the enzyme is produced by the microorganism in an amount that is 2-fold greater than that produced by a human cell line. The term “preferentially expressed” means that the enzyme is produced by the microorganism in an amount that is 10-fold greater than that produced by a human cell line. The term “exclusively expressed” means that the enzyme is produced by the microorganism in an amount that is 100-fold greater than that produced by a human cell line.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Compounds and Synthesis

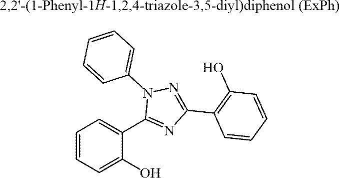

Phenylhydrazine (2.30 mL, 23.40 mmol) was added to a suspension of 2-(2-hydroxyphenyl)-4H-benzo[e][1,3]oxazin-4-one (Pramanik et al., 2015) (2.80 g, 11.70 mmol) in Et₂O (30 mL) and the mixture was heated to reflux for 12 hrs. The reaction mixture was cooled to room temperature and the solvent was removed under reduced pressure. The crude product was triturated (MeOH) and the white solid was isolated by filtration to afford the title product as a white solid (1.85 g, 5.62 mmol, 48%). ¹H NMR (500 MHz, CDCl₃) δ 11.66 (s, 1 H), 9.69 (s, 1 H), 8.15 (dd, J = 1.4, 7.8 Hz, 1 H), 7.67 - 7.56 (m, 3 H), 7.55 - 7.50 (m, 2 H), 7.41 - 7.30 (m, 2 H), 7.16 - 7.03 (m, 3 H), 6.93 (dd, J = 1.1, 8.1 Hz, 1 H), 6.64 (t, J = 7.6 Hz, 1 H); ¹³C NMR (126 MHz, CDCl₃) δ 159.0, 158.2, 156.5, 152.0, 138.2, 132.8, 131.7, 130.3, 130.0, 127.6, 127.4, 126.4, 119.8, 118.8, 118.3, 117.1, 113.4, 110.1; HRMS: m/z calculated for C₂₀H₁₅N₃O₂: requires 330.1237 for [M+H]⁺, found 330.1244. All experimental data matches the data reported in the literature (Dahm et al., 2015).

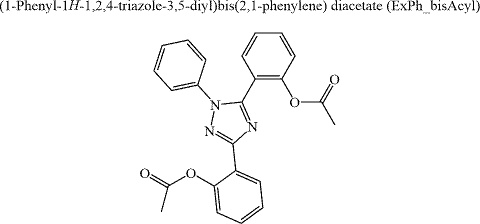

4-Dimethylaminopyridine (0.05 g) was added to a suspension of ExPh (1.00 g, 3.04 mmol) in Ac₂O (10 mL) and the resultant solution was left to stir overnight. The solvent was removed under reduced pressure and the crude mixture was purified via column chromatography 20:80 (EtOAc: petroleum ether) to afford the title product as a white solid (0.60 g, 1.45 mmol, 48%).¹H NMR (400 MHz, CDCl₃) δ 8.39 (dd, J = 1.8, 7.8 Hz, 1 H), 7.52 - 7.34 (m, 8 H), 7.33 - 7.15 (m, 4 H), 2.35 (s, 3 H), 2.09 (s, 3 H); ¹³C NMR (126 MHz,CDCl₃) δ 170.5, 168.9, 158.9, 150.4, 148.8, 148.4, 137.9, 131.5, 131.3, 130.5, 129.8, 129.3, 128.6, 126.4, 125.9, 124.1, 123.7, 123.3, 121.5, 21.4, 20.9; HRMS: m/z calculated for C₂₄H₁₉N₃O₄: requires 414.1448 for [M+H]⁺, found 414.1453

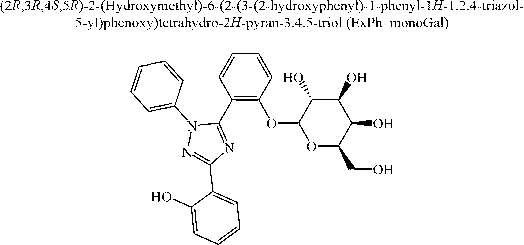

To a mixture of ExPh (0.50 g, 1.51 mmol), Cs₂CO₃ (2.00 g, 6.04 mmol), Na₂SO₄ (0.86 g, 6.04 mmol) in DMF (10 mL), 2,3,4,6-tetra-O-acetyl-α-D-galactopyranosyl bromide (2.50 g, 6.04 mmol) was added. The reaction mixture was left to stir overnight and another portion of Cs₂CO₃ (2.00 g, 6.04 mmol) and 2,3,4,6-tetra-O-acetyl-α-D-galactopyranosyl bromide (2.50 g, 6.04 mmol) were added. The reaction mixture was left to stir overnight and H₂O (50 mL) were added. The aqueous layer was extracted with EtOAc (3 × 50 mL) and the combined organics were washed with brine (3 × 50 mL), dried (MgSO₄) and solvent was removed in vacuo. The reaction mixture was purified via column chromatography 20:80 to 50:50 (EtOAc:hexanes) to afford a mixture of desired product and excess 2,3,4,6-tetra-O-acetyl-α-D-galactopyranosyl bromide. The crude mixture was dissolved in MeOH and K₂CO₃ (5.00 g) was added. The reaction mixture was stirred for 2 hours before being filtered and have the solvent removed under reduced pressure. The crude mixture was purified via column chromatography 50:50 to 100:0 (EtOAc:hexanes) to afford the title compounds as a white solid (0.13 g, 0.264 mmol, 18%). ¹H NMR (400 MHz, CD₃OD) δ 8.08 (dd, J = 1.4, 7.8 Hz, 1 H), 7.53 - 7.45 (m, 3 H), 7.44 - 7.38 (m, 3 H), 7.38 - 7.29 (m, 3 H), 7.09 (dt, J = 1.0, 7.5 Hz, 1 H), 7.00 - 6.93 (m, 2 H), 4.90 (d, J = 7.6 Hz, 1 H), 3.85 (d, J = 0.8 Hz, 1 H), 3.70 (s, 2 H), 3.65 - 3.59 (m, 2 H), 3.54 - 3.49 (m, 1 H); ¹³C NMR (126 MHz, CDCl₃) δ 160.9, 156.7, 156.3, 151.0, 137.1, 132.6, 131.5, 131.2, 129.5, 129.0, 127.2, 124.6, 123.2, 119.9, 117.9, 117.7, 117.2, 113.9, 104.6, 75.0, 73.1, 71.3, 69.0, 62.5, 50.9; HRMS: m/z calculated for C₂₆H₂₅N₃O₇: requires 492.1765 for [M+H]⁺, found 492.1767

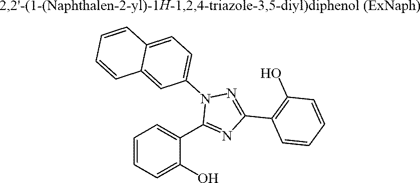

2-Napthylhydrazine hydrochloride (4.56 g, 23.40 mmol) was added to a suspension of 2-(2-hydroxyphenyl)-4H-benzo[e][1,3]oxazin-4-one (Pramanik et al., 2015) (2.80 g, 11.70 mmol) in PhMe (20 mL) and the mixture was heated to reflux for 12 hrs. The reaction mixture was cooled to room temperature and the solvent was removed under reduced pressure. The crude product was triturated (MeOH) and the white solid was isolated by filtration to afford the title product as a white solid (2.55 g, 6.72 mmol, 57%). ¹H NMR (500 MHz, CDCl₃) δ 8.18 (dd, J = 1.2, 7.6 Hz, 1 H), 8.11 - 7.97 (m, 3 H), 7.93 (d, J = 7.6 Hz, 1 H), 7.72 - 7.61 (m, 2 H), 7.55 (dd, J = 1.8, 8.5 Hz, 1 H), 7.44 - 7.36 (m, 1 H), 7.36 - 7.29 (m, 1 H), 7.16 (d, J = 8.2 Hz, 1 H), 7.13 - 7.04 (m, 2 H), 6.97 (d, J = 7.9 Hz, 1 H), 6.57 (t, J = 7.6 Hz, 1 H); ¹³C NMR (126 MHz, CDCl₃) δ 159.1, 158.3, 156.6, 152.1, 135.4, 133.5, 133.2, 132.9, 131.7, 130.2, 128.5, 128.1, 127.9, 127.7, 127.6, 127.6, 125.4, 123.5, 119.9, 118.9, 118.3, 117.2, 113.4, 110.1; HRMS: m/z calculated for C₂₄H₁₇N₃O₂: requires 380.1394 for [M+H]⁺, found 380.1405

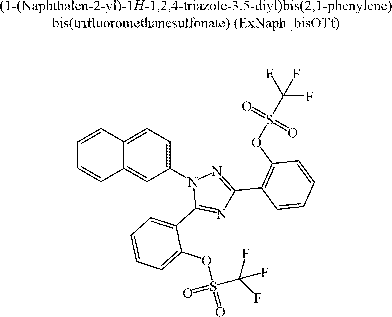

A solution of ExNaph (0.50 g, 1.32 mmol) and pyridine (1.60 mL, 19.80 mmol) in DCM (10 mL) was cooled to 0° C. and trifluoromethanesulfonic anhydride (2.22 mL, 13.20 mmol) was added dropwise. The reaction mixture was left to stir overnight. The solvent was removed under reduced pressure and the crude mixture was purified via column chromatography 20:80 to 50:50 (DCM:hexanes) to afford the title compound as a white solid (0.32 g, 0.50 mmol, 38%). ¹H NMR (500 MHz, CDCl₃) δ 8.48 - 8.41 (m, 1 H), 7.92 (d, J= 1.8 Hz, 1 H), 7.91 - 7.87 (m, 2 H), 7.79 (d, J = 7.6 Hz, 1 H), 7.62 - 7.50 (m, 7 H), 7.44 - 7.37 (m, 3 H); ¹³C NMR (126 MHz, CDCl3) δ 158.5, 149.3, 147.2, 147.0, 134.7, 133.0, 132.9, 132.4, 132.3, 131.3, 130.9, 129.5, 128.7, 128.6, 128.4, 127.9, 127.2, 127.1, 124.7, 123.0, 122.8, 122.6, 122.5, 121.9, 120.1, 119.7, 117.5, 117.2; ¹⁹F NMR (471 MHz, CDCl₃) δ - 73.65, - 73.38; HRMS: m/z calculated for C₂₆H₁₅F₆N₃O₆S₂: requires 644.0379 for [M+H]⁺, found 644.0382.

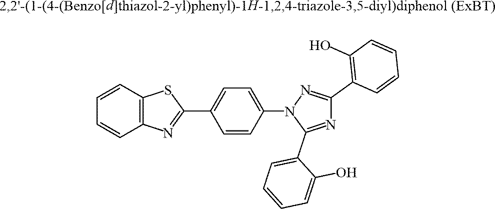

A mixture of ExJade (5.60 g, 15.00 mmol), 2-aminothiophenol (1.56 mL, 15.00 mmol), tetrabutylammonium bromide (4.80 g, 15.00 mmol) and triphenylphosphite (3.90 mL, 15.00 mmol) was heated to 120° C. for 2 hours. The reaction mixture was cooled to room temperature and the crude material was triturated (MeOH). The white solid was isolated via filtration to afford the title compound (3.45 g, 7.45 mmol, 50%). ¹H NMR (500 MHz, CDCl₃) δ 8.35 - 8.28 (d, J = 8.5 Hz, 2 H), 8.20 - 8.09 (m, 2 H), 7.98 (d, J = 7.9 Hz, 1 H), 7.70 - 7.65 (d, J = 8.5 Hz, 2 H), 7.57 (t, J = 7.5 Hz, 1 H), 7.48 (s, 1 H), 7.43 - 7.35 (m, 2 H), 7.17 (d, J = 8.2 Hz, 1 H), 7.12 - 7.04 (m, 3 H), 6.70 (t, J = 7.6 Hz, 1 H); ¹³C NMR (126 MHz, CDCl₃) δ 165.8, 159.5, 158.1, 156.6, 154.1, 152.2, 139.8, 135.3, 135.2, 133.1, 131.9, 129.0, 127.7, 127.6, 126.8, 125.9, 123.6, 121.8, 119.9, 119.1, 118.5, 117.2, 113.2, 110.0; HRMS: m/z calculated for C₂₇H₁₈N₄O₂S: requires 463.1223 for [M+H]⁺, found 463.1224

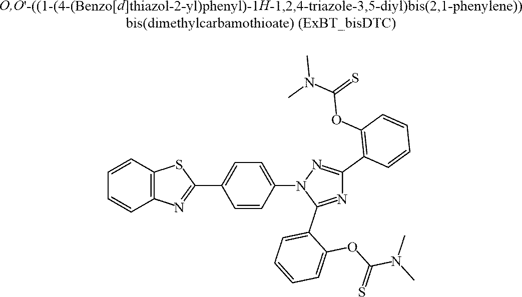

A solution of ExBT (0.50 g, 1.08 mmol), dimethylthiocarbamoyl chloride (1.40 g, 10.8 mmol), 4-dimethylaminopyridine (0.05 g), NEt₃ (2.20 mL, 16.23 mmol) in THF was refluxed for three days. The solvent was removed in vacuo and the reaction mixture was purified via column chromatography 50:50 (DCM:hexanes) to 20:80 (EtOAc:hexanes) to afford the title compound as a silver-like foam (0.26 g, 0.41 mmol, 38%). ¹H NMR (500 MHz, CDCl₃) δ 8.36 (dd, J = 1.4, 7.8 Hz, 1 H), 8.17 - 8.08 (m, 3H), 7.94 (d, J = 7.9 Hz, 1 H), 7.77 (d, J = 8.5 Hz, 2 H), 7.57 - 7.48 (m, 3 H), 7.47 - 7.38 (m, 2 H), 7.33 (d, J = 8.2 Hz, 1 H), 7.26 - 7.15 (m, 3 H), 3.44 (s, 3 H), 3.34 (s, 3 H), 3.30 (s, 3 H), 3.27 (s, 3 H); ¹³C NMR (126 MHz, CDCl₃) δ 187.6, 186.1, 166.7, 159.1, 153.5, 152.3, 151.6, 149.9, 139.8, 134.9, 132.9, 131.4, 131.0, 130.2, 129.6, 128.3, 126.8, 126.2, 125.9, 125.8, 125.1, 125.0, 124.6, 123.9, 123.2, 122.3, 121.8, 43.3, 39.1; HRMS: m/z calculated for C₃₃H₂₈N₆O₂S₃: requires 637.1509 for [M+H]⁺, found 637.1517

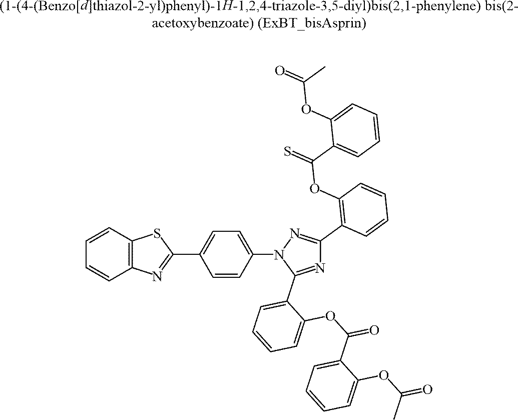

A solution of ExBT (0.50 g, 1.08 mmol) and pyridine (0.87 mL, 10.80 mmol) in THF was cooled to 0° C. 2-(Chlorocarbonyl)phenyl acetate (Pathak and Dhar, 2015) (1.10 g, 5.54 mmol) was added and the reaction mixture was stirred at 0° C. and monitored by TLC. The reaction remained at 0° C. for 5 hours and another portion of 2-(Chlorocarbonyl)phenyl acetate (1.10 g, 5.54 mmol) was added. Once starting material was consumed (determined by TLC), the reaction was quenched with H₂O (50 mL). The aqueous layer was extracted with EtOAc (3 × 50 mL) and the combined organics were washed with brine (3 × 50 mL), dried (MgSO₄) and solvent was removed in vacuo. The reaction mixture was purified via column chromatography 20:80 (EtOAc:hexanes) to afford the title compound as a white solid ( 0.37 g, 0.47 mmol, 44 %). ¹H NMR (400 MHz, CDCl₃) δ 8.40 (dd, J = 1.6, 7.8 Hz, 1 H), 8.20 (dd, J = 1.6, 7.8 Hz, 1 H), 8.09 (d, J = 8.0 Hz, 1 H), 7.93 (d, J = 7.6 Hz, 1 H), 7.90 (dd, J = 1.5, 7.9 Hz, 1 H), 7.79 -7.74 (m, J= 8.6 Hz, 2 H), 7.64 - 7.46 (m, 5 H), 7.45 - 7.32 (m, 4 H), 7.30 - 7.20 (m, 4 H), 7.18 (d, J = 8.2 Hz, 1 H), 7.16 - 7.11 (m, J = 8.6 Hz, 2 H), 7.09 - 7.05 (m, 1 H), 2.26 (s, 3 H), 2.10 (s, 3H); ¹³C NMR (126 MHz, CDCl₃) δ 169.7, 169.5, 166.9, 163.2, 161.8, 159.0, 153.2, 151.5, 151.1, 150.3, 148.4, 148.2, 139.6, 134.6, 134.2, 133.2, 132.2, 131.8, 131.5, 130.6, 130.0, 128.1, 126.8, 126.4, 126.3, 126.0, 125.9, 125.8, 124.0, 123.9, 123.7, 123.5, 123.4, 123.1, 123.0, 121.8, 121.7, 121.5, 21.0, 20.8; HRMS: m/z calculated for C₄₅H₃₀N4O₈S₃: requires 787.1857 for [M+H]⁺, found 787.1857

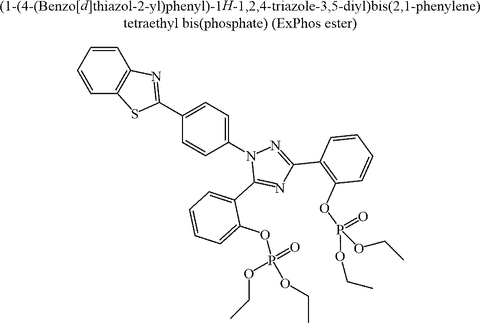

Diethyl chlorophosphate (1.56 mL, 10.81 mmol) was added dropwise to a solution of ExBT (0.50 g, 1.08 mmol), DMAP (0.05 g, Cat.) and NEt₃ (1.45 mL, 10.81 mmol) in THF (10 mL). The reaction mixture was stirred at room temperature for 16 hours and upon completion the solvent was removed in vacuo. H₂O (50 mL) and EtOAc (100 mL) was then added and the organic layer was washed with H₂O (3 × 50 mL), brine (50 mL), dried (MgSO₄) and concentrated under reduced pressure to afford the crude product. The crude material was purified via column chromatography 10:90 (EtOAc/petroleum ether) to 50:50 (EtOAc/petroleum ether) and triturated (Et₂O) to afford the title compound as a white solid (0.23 g, 0.31 mmol, 29%). ¹H NMR (500 MHz, CDCl₃) δ 8.22 (d, J = 7.9 Hz, 1 H), 8.14 - 8.06 (m, 3 H), 7.93 (d, J = 7.9 Hz, 1 H), 7.64 - 7.40 (m, 9 H), 7.30 (t, J = 7.5 Hz, 2 H), 4.27 (m, 4 H), 4.00 - 3.86 (m, 4 H), 1.24 (t, J = 7.0 Hz, 6 H), 1.16 (t, J = 7.0 Hz, 6 H); ¹³C NMR (126 MHz, CDCl₃) δ 166.3, 159.6, 154.0, 150.7, 148.6, 148.6, 140.0, 135.1, 133.2, 132.2, 131.6, 130.7, 126.6, 125.6, 125.4, 125.0, 123.9, 123.4, 122.3, 122.3, 121.7, 121.0, 120.9, 120.8, 120.8, 64.7 (C-P, J = 6.36 Hz) 16.1(C-P, J = 6.36 Hz), 15.9 C-P, J = 7.27 Hz); ³¹P NMR (202 MHz, CDCl₃) δ -6.87, - 7.11; HRMS: m/z calculated for C₃₅H₃₆N₄O₈P₂S: requires 735.1802 for [M+H]⁺, found 735.1812

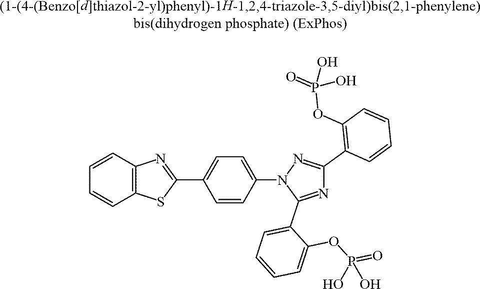

A solution of ExPhos ester (0.20 g, 0.27 mmol) in dry DCM (5 mL) was cooled to 0° C. and bromotrimethylsilane (0.36 mL, 2.70 mmol) was added dropwise. The reaction mixture was allowed to warm to room temperature and the reaction was monitored by LC-MS. Upon completion (~ 48 h), the solvent was removed under reduced pressure and MeOH (10 mL) was added and the solution was left to stir for 1 hour. The solvent was then removed under reduced pressure and the crude material was purified by trituration (1. Et₂O 2. DCM) to afford the title compound as a cream solid (0.11 g, 0.17 mmol, 65%). ¹H NMR (500 MHz, CD₃OD) δ 8.16 -8.10 (d, J = 8.5 Hz, 2 H), 7.97 - 7.91 (m, 3 H), 7.71 - 7.67 (d, J = 8.9 Hz, 2 H), 7.67 - 7.56 (m, 3H), 7.50 - 7.44 (m, 2 H), 7.42 - 7.35 (m, 3 H), 7.32 (t, J = 7.6 Hz, 1 H) ¹³C NMR (126 MHz, CD₃OD) δ 166.5, 153.6, 153.1, 149.6, 149.5, 149.4, 149.3, 138.1, 134.9, 134.7, 134.7, 133.2, 131.6, 130.8, 128.3, 126.8, 126.0, 125.6, 125.4, 125.4, 122.6, 122.5, 122.1, 121.9, 118.3, 115.4; ³¹P NMR (202 MHz, CD₃OD) δ -5.24, -5.67; HRMS: m/z calculated for C₂₇H₂₀N₄O₈P₂S: requires 623.0550 for [M+H]⁺, found 623.0555

### Example 2: Biological Activity

### I. Screening Assay

Cell culture. A549 cells (ATCC® CCL-185™) were maintained in growth medium consisting of RPMI 1640 Medium (Sigma-Aldrich, MO, USA) supplemented with 10% fetal bovine serum (Sigma-Aldrich, MO, USA) and 2% Penicillin/Streptomycin (Sigma-Aldrich, MO, USA). Cells were kept in a humidified atmosphere of 5% CO₂ and 95% air at 37° C. and were split when they reached 90% confluency.

Cell viability assay. 1500 cells per well were plated on a 96-well plate (12 rows × 8 wells per row) in 100 µL growth medium and kept at 37° C. under 5% CO₂. 24 h after seeding, cells were treated with another 100 µL growth medium containing different concentrations of the drug under evaluation. Cells in each row of the 96-well plate were treated with the same concentration of drug and the first two rows were kept as control and treated with growth medium alone. The highest drug concentration evaluated was 250 µM and lower concentrations were produced via serial dilution by a factor of 3. Treated cells were kept at 37° C. under 5% CO₂ for 72 h. Then, 50 µL RPMI 1640 Medium without fetal bovine serum and penicillin/streptomycin containing 3 mg/mL 3-(4,5-dimethylthiazol-2-yl)-2,5-diphenyltetrazolium bromide (Sigma-Aldrich, MO, USA) was added to each well and cells were kept at 37° C. under 5% CO₂ for another 4 h. Subsequently, the medium was removed, and the precipitated formazan dye taken up in DMSO (50 µL). The absorbance of the solutions was measured at 540 nm with an M5 microplate reader (Molecular Devices, USA) and 0.33 absorbance units were subtracted from all wells (this absorbance value corresponds to wells containing DMSO without dye - control). The absorbance of each row of wells were averaged and were directly proportional to the number of viable cells after drug treatment.

### II. Anti-Microorganism Properties and Toxicity

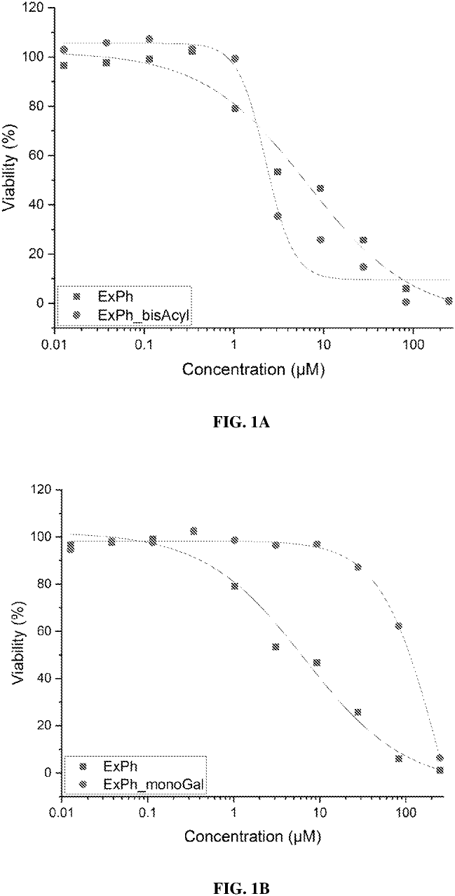

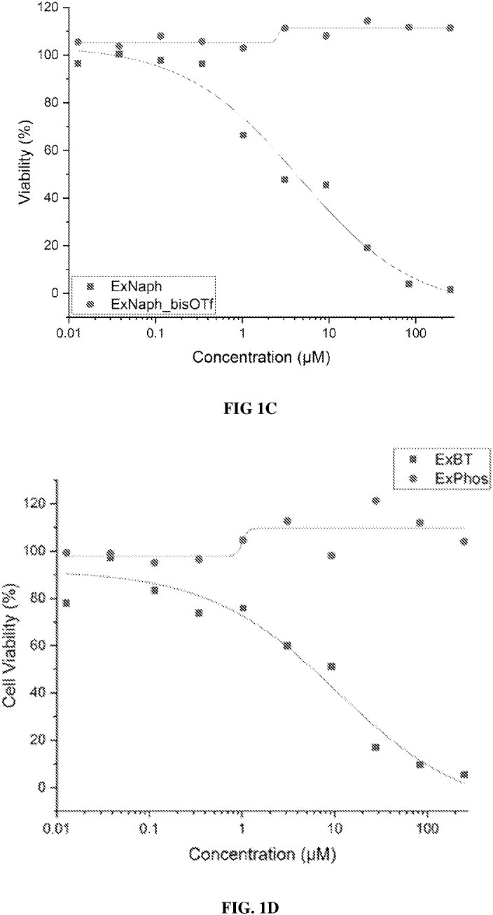

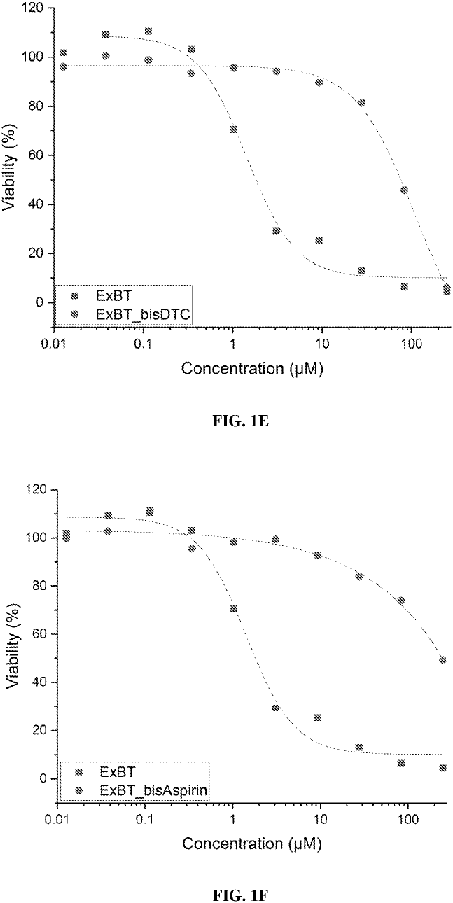

Results of toxicity comparing the protected and unprotected derivatives are shown in FIGS. 1A-1F.

Similarly, the bacterial inhibitory effect of the present ExJade derivatives was then evaluated. Initial tests were focused on two Gram-negative, ESKAPE bacteria (Mulani et al., 2019), Pseudomonas aeruginosa (ATCC 27853) and Klebsiella pneumoniae (ATCC 13883). Arbekacin, an FDA-approved aminoglycoside-based antibiotic, was used as positive control. ExJade, ExPh, ExBT, and ExPhos were found capable of inhibiting the growth of these two bacterial strains in a statistically significant manner.

More importantly, two clinically relevant gram-positive, drug-resistant bacteria, methicillin-resistant Staphylococcus aureus (MRSA, ATCC 43300) and vancomycin-resistant Enterococcus faecalis (VRE, ATCC 51299) with high ALP activity were then used to evaluate further the therapeutic potential of these ExJade derivatives. Again, arbekacin was used as a positive control. Strong inhibitory activities were observed for the suppression of these “superbugs” with a potency comparable to that of arbekacin (FIG. 4). The observed inhibition of VRE is of particular significance since there are few clinically available treatments for this multidrug-resistant bacterium.

The pre-incubation with increasing concentrations of Fe(III) led to a corresponding decrease in the inhibitory activity including no appreciable inhibitory activity seen for either MRSA or Pseudomonas aeruginosa in the case of the control system ExBT-OMe. This observation confirms the Fe(III) chelation dependent inhibitory activity. Due to the Fe(III) chelation dependent nature of the ExJade derivatives and to illustrate their potential “real life” utility, the inhibitory activity of ExBT and ExPhos against MRSA were evaluated in a blood containing medium consisting of agar and defibrillated amniotic blood (Filipowicz et al., 2003). Remarkably, no adverse effect was seen, with the inhibitory activity of ExBT and ExPhos remaining strong. In addition, no significant cytotoxicity was observed for ExPhos towards A549 cells, providing an initial indication that the ExJade-based pro-chelation approach reported here may prove selective towards bacteria.

Lastly, to determine whether the present ExJade derivatives might have a role to play as fluorescence imaging agents and theranostic agents, MRSA was incubated separately with ExBT and ExPhos; both compounds produced bright fluorescence in bacteria (FIG. 5A) Interestingly, the pre-treatment of MRSA with Phosphatase Inhibitor cocktail A (sodium fluoride, sodium pyrophosphate, β-glycerophosphate and sodium orthovanadate) led to an inhibitor concentration dependent decrease in the fluorescence emission intensity (FIG. 5A and FIG. 5B). This finding further confirms the contention that ExPhos is a pro-chelator that is activated under conditions of use via a phosphatase-mediated pathway.

### II. Screening for Fluorescence

Several new ExJade derivatives, designed to control solubility and modulate the intrinsic electronic features, namely ExPh, ExPh-OMe, ExH, ExBT, ExBT-OMe and ExPhos, were prepared. Two of these derivatives, ExPh and ExBT, were found to display aggregation induced emission (AIE)-like properties in aqueous media (FIG. 2) (Hong et al., 2009; Qian and Tang, 2017). Specifically, insoluble aggregates (as confirmed by DLS) formed in water, leading to a strong fluorescence emission with a large Stokes shift (> 150 nm). Single crystal X-ray diffraction (XRD) analysis for ExPh and ExBT revealed strong intramolecular and intermolecular hydrogen bonding interactions, which is indicative of an excited state proton transfer based system (FIGS. 2A & 2B) (Sedwick et al., 2018; Zhao et al., 2015; Hao and Chen, 2016; Tang et al., 2011; Peng et al., 2015; Sahu et al., 2018; Das et al., 2019; Sedgwick et al., 2018; Wu et al., 2018). Further analysis of the crystal packing diagram revealed that both ExPh and ExBT exists in a slip-stacking orientation, which is known to augment rigidity in AIEgens since it serves to restrict intramolecular rotation (RIR) and enhance the fluorescence-based emission intensity (Lou et al., 2018; Sivalingam et al., 2109). In marked contrast to what is seen in aqueous environments, solutions of ExPh and ExBT in most common organic solvents display no appreciable emission.

In an attempt to elucidate in greater detail the origins of the fluorescence displayed by ExPh and ExBT under conditions favoring aggregation, the derivatives, ExPh-OMe, ExBT-OMe and non-N phenyl functionalized ExH, were studied (Sahu et al., 2018; Das et al., 2019). In both, aqueous and organic media, ExH was found to display strong fluorescence. This led us to believe the free rotation of the Ph unit effectively quenches the excited state fluorescence of ExPh and ExBT, a phenomenon known as the free rotor effect (Lou et al., 2018). Upon blocking each phenol unit present on ExPh and ExBT with a methyl group (to give ExPh-OMe and ExBT-OMe), a significant blue-shift in the emission maximum was seen. Such changes in the emission features are consistent with the contention that the free form of the phenol is required for efficient ESIPT (Sedgwick et al., 2018).

No significant fluorescence quenching was seen upon changing the solvents in which ExPh-OMe and ExBT-OMe were analyzed - DMSO, PhMe, THF, DCM, acetone, MeCN, MeOH and PBS. On this basis we suggest that the ESIPT process is heavily affected by the free rotor effect (Vollmer and Rettig 1996; Kim et al., 2007). In addition, significant solvatochromism was observed for both ExPh-OMe and ExBT-OMe, which leads us to suggest that a type of intramolecular charge transfer (ICT) process (Pannipara et al., 2015) is additionally involved in mediating the fluorescence features of these ExJade derivatives. This putative ICT phenomenon has been observed in structurally similar N-phenylpyrrole analogues (Yoshihara et al., 2005; Yoshihara et al., 2003). Further support for the proposed aqueous induced aggregation mechanism invoked in the case of ExPh and ExBT-OMe came from studies of the parent system, ExJade. In the solid-state ExJade fluoresces; (Zhang et al., 2019) however, it does not do so in aqueous solution, presumably as the result of its good solubility at neutral pH. In the event, ExPh and ExBT proved to be highly emissive in aqueous media, making these species of interest in terms of the potential optical imaging of bacteria and other organisms.

The inherent fluorescence of ExJade derivatives were contemplated to provide a unique multifunctional “molecular platform” that can be used to develop a fluorescence responsive pro-chelator active against antibiotic resistant bacteria. This system permits both detection and treatment with little synthetic investment. As a result, this strategy was explored using ExBT, which displayed optical characteristics deemed suitable for fluorescence imaging (λₑₓ > 350 nm). Phosphate is an essential nutrient for bacterial growth and phosphatases are expressed in a range of bacteria (Sajid et al., 2015; Braiband et al., 2001; Kriakov et al., 2003). ExPhos was thus developed to act as a phosphatase-responsive pro-chelator active against antibiotic resistant bacteria with minimal off-target toxicities. The pro-chelator function was not expected to be manifest in the case of the control system, ExBT-OMe.

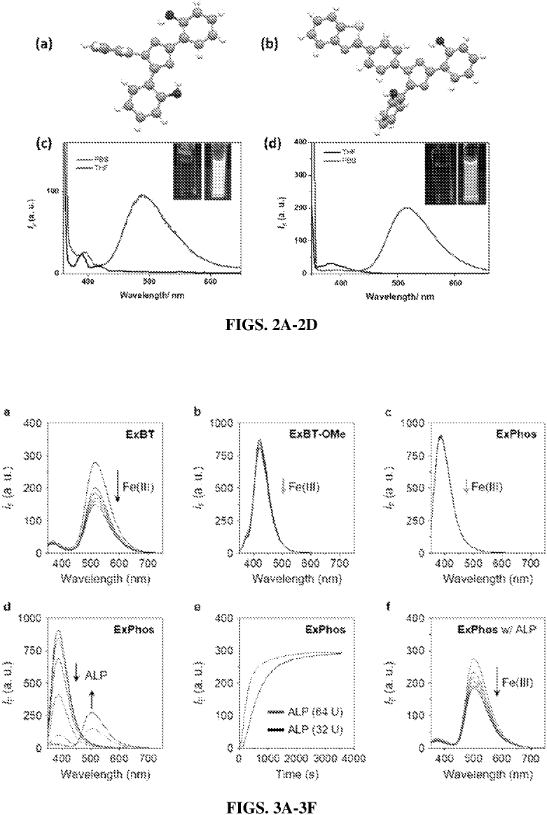

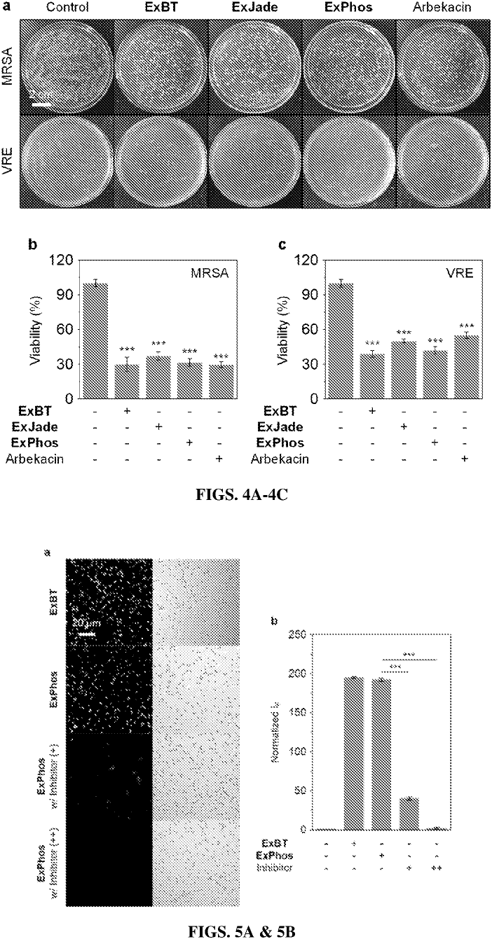

As expected and previously seen for ExJade, (Steinhauser et al., 2004) the addition of increasing concentrations of Fe(III) (as the FeCl₃ salt) to both ExBT (15 µM) and ExJade (15 µM) led to a gradual increase in their respective UV-Vis absorption intensities, along with a color change from clear to purple. Fluorescence experiments revealed a concentration-dependent quenching of the ExBT fluorescence (FIG. 3A), a common observation for Fe(III) chelation (Qu et al., 2012). No change in the UV-vis absorption or fluorescence was seen for either ExBT-OMe or ExPhos (FIGS. 3B & 3C). This is consistent with the design expectation that a free phenol is need to chelate Fe(III) well (Steinhauser et al., 2004)

In the presence of alkaline phosphatase (ALP), a dose dependent change in fluorescence emission was observed (0 - 64 U), with a final fluorescence emission profile analogous to that of ExBT (FIG. 3D). This ratiometric change in fluorescence emission was ascribed to the gradual and stepwise dephosphorylation of ExPhos, which was confirmed by LC-MS analysis. ExPhos solutions exposed to ALP (64 U ALP) were subsequently treated with increasing concentrations of Fe(III) (as the FeCl₃ salt). As expected for a system capable of chelating Fe(III), a quenching of the fluorescence intensity was observed (FIG. 3F). Based on these findings, we considered it likely that ExPhos could serve as an ALP-responsive Fe(III) pro-chelator.

### Example 3: Photophysical Properties of Deferasirox (ExJade) and Derivatives

### I. Introduction

Deferasirox (ExJade) is an FDA-approved iron(III) chelator used for the treatment of iron overload (Moukalled et al., 2017; Shirley and Plosker, 2014; Yang et al., 2007). Iron is essential for most living organisms and plays a critical role in numerous human physiological and pathological processes (Jung et al., 2019), including bacterial growth and biofilm formation (Oh et al., 2018; Cassat and Skaar, 2013; Lin et al., 2012). This has led to explorations of deferasirox in the context of new therapeutic applications. Recent studies have demonstrated the utility of deferasirox as chemotherapeutic (Tury et al., 2018; Kielar et al., 2012), antifungal (Puri et al., 2019), and antimicrobial agents (Moon et al., 2013; Moreau-Marquis et al., 2009; Post et al., 2019). However, deferasirox is not inherently luminescent and has not been extensively explored as a potential diagnostic. This example reports that the deferasirox scaffold acts as an aggregationinduced emission luminogen (AIEgen) with certain derivatives displaying a seemingly unique combination of aggregationinduced emission (AIE), excited state intramolecular proton transfer (ESIPT), charge transfer (TICT), and through-bond and through-space conjugation characteristics. The diagnostic potential of several new deferasirox derivatives was shown in bacterial biofilm studies, which revealed antibiofilm activity equal to the nonemissive deferasirox parent.

### II. Results

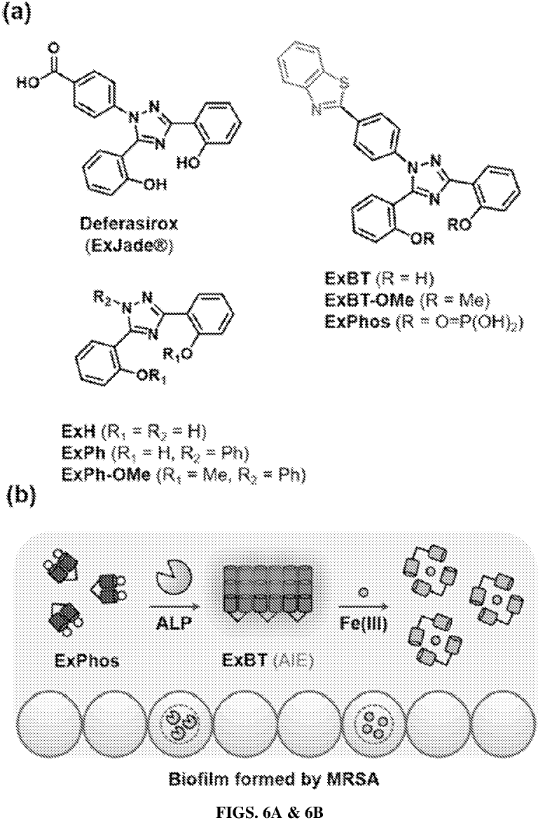

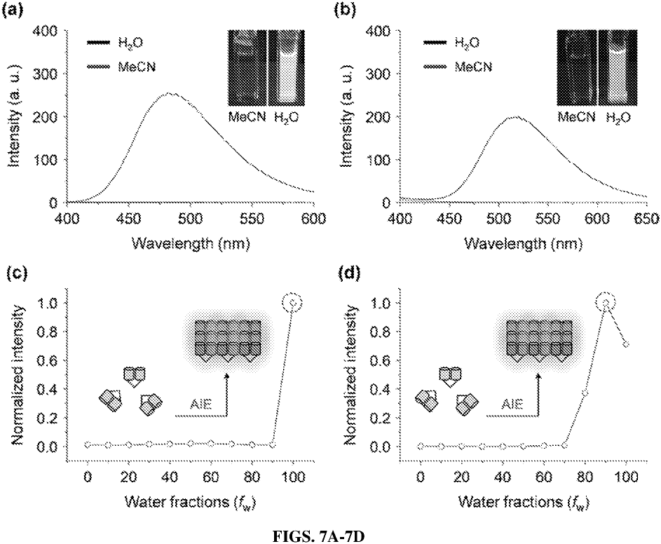

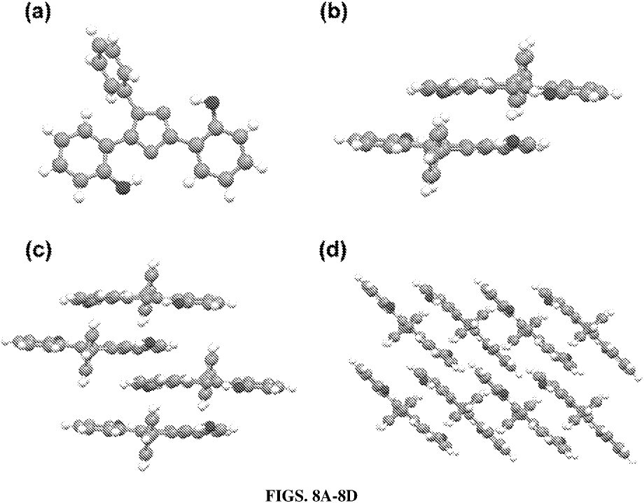

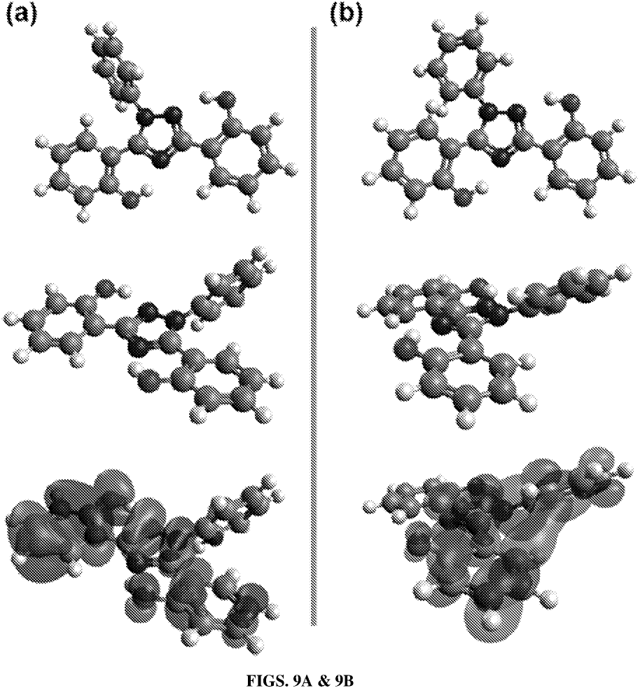

Deferasirox and its derivatives used in this study, ExPh, ExPh-OMe, ExBT, ExBT-OMe, and the fluorescent responsive pro-chelator ExPhos, are shown in FIG. 6. Initially, the focus was on the synthesis of deferasirox derivatives as chemotherapeutics (Steinbrueck et al., 2020). Unexpectedly, ExPh and ExBT proved emissive in aqueous media; however, in organic solution, they were found to be nonfluorescent. This observation is attributed to the fluorescence phenomenon known as AIE, wherein ExPh and ExBT form insoluble fluorescent aggregates in water (FIGS. 7A & 7B) and confirmed by DLS. Studies in mixed MeCN-aqueous media revealed appreciable emission when the water fraction was >80% (FIGS. 7C & 7D). Most reported AIEgens (systems giving rise to AIE) are propeller-like analogues of tetraphenylethylene (TPE) or that can rotate freely in solution (Yang et al., 2018; Chen et al., 2019; Dou et al., 2019). Neither ExPh nor ExBT share this structural motif (Zhang et al., 2019).

Single-crystal X-ray diffraction analyses of ExPh and ExBT revealed intramolecular and intermolecular hydrogen-bonding interactions in the solid state (FIG. 8). Analysis of the packing diagrams for ExPh and ExBT revealed a slip-stack orientation. These findings led consideration of if an excited state intramolecular proton transfer combined with an AIE effect (ESIPT+AIE) might be responsible for the enhanced emission seen in aqueous media (Sedgwick et al., 2018). However, AIE +ESIPT fluorophores can display fluorescence emission in both organic and aqueous solvents (Sedgwick et al., 2018; Liu et al., 2017; Peng et al., 2017). In contrast, ExPh and ExBT displayed no appreciable fluorescence intensity in organic solution (cf. FIGS. 8C & 8D). This finding and the different fluorescent maxima seen for ExPh (λₘₐₓ = 480 nm) and ExBT (λₘₐₓ = 530 nm) in aqueous media led us to consider a more complex mechanism.

Toward this end, the fluorescent properties of the previously reported phenyl-free ExH were studied (Sahu et al., 2018; Das et al., 2019). In contrast to ExPh and ExBT, ExH was found to be strongly fluorescent in both aqueous and organic media. It is thus not an AIEgen. Next, the phenol units present in ExPh and ExBT were blocked with methyl groups to give ExPh-OMe and ExBT-OMe. This modification produced a significant blue-shift in the emission maxima. However, it also led to a loss in the AIE-like properties. From this data, it was concluded that hydrogen-bonding interactions play a role in promoting the formation of fluorescent aggregates in the case of ExPh and ExBT. In contrast to ExPh and ExBT, solvent-dependent behavior was seen for both ExPh-OMe and ExBT-OMe. On this basis it was suggested that intramolecular charge transfer/twisted intramolecular charge transfer (ICT/TICT) (Sasaki et al., 2016; Tian et al., 2021) processes modulate the fluorescence features of the deferasirox core.

Next, ground and excited state calculations were carried out using DFT and time-dependent density functional theory (TDDFT), respectively. Several functionals were screened in the case of ExPh. The long-range corrected potentials CAMB3LYP and ωB97XD best matched the experimental UV-vis spectral data and were thus chosen for subsequent calculations. Excited state natural transition orbital (NTO) calculations were performed on the electronically excited singlet state (S1). Vertical excitation of the deferasirox derivatives, ExPh, ExBT, ExPh-OMe, and ExBTOMe, results in a change in the electron density consistent with charge transfer. Geometry optimization of the S1 excited state revealed a planarization between the phenyl substituent and the triazole core, as expected for systems subject to TICT. Subsequent calculations on the keto tautomers were performed to determine the propensity to undergo ESIPT. In the case of ExPh, the excited keto form lies at higher energy (+9.6 eV) than the more stable TICT enol form. In contrast, ExH, a reported ESIPT system, was characterized by an excited keto form that was more stable (-0.8 eV) than the corresponding enol form (Sahu et al., 2018). This is consistent with ExH being fluorescent in both polar and apolar solution in contrast to ExPh, which favors TICT. More nuanced behavior was seen in the case of ExBT; here a small energy difference (+0.14 eV) between the excited keto and TICT enol tautomers was seen, presumably as the result of the benzothiazole unit. Thus, both TICT and ESIPT mechanisms need to be considered. NTO calculations revealed additional through-space conjugation (TSC) between the phenol and phenyl unit in ExPh, ExPh-OMe, and ExBT, an effect that can be enhanced through aggregation (FIG. 9) (He et al., 2016; Wang et al., 2015; Shen et al., 2019).

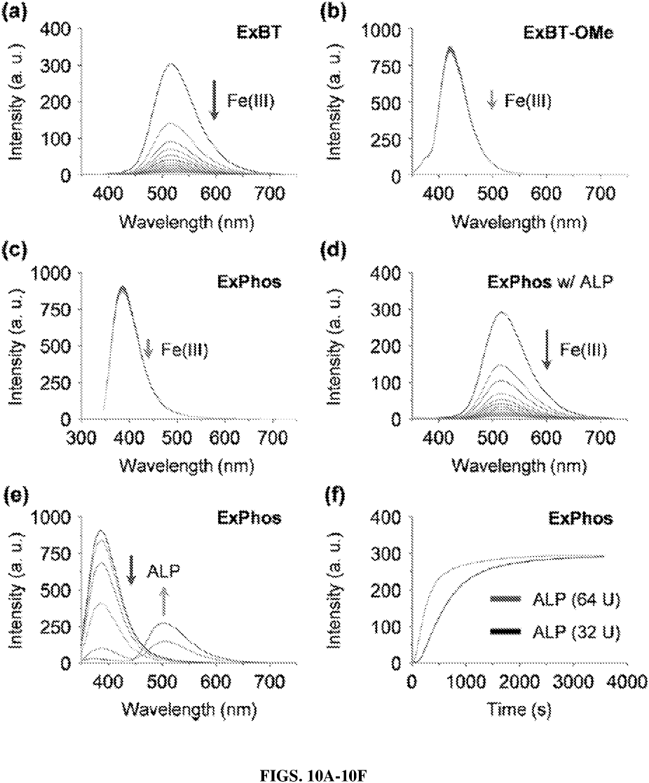

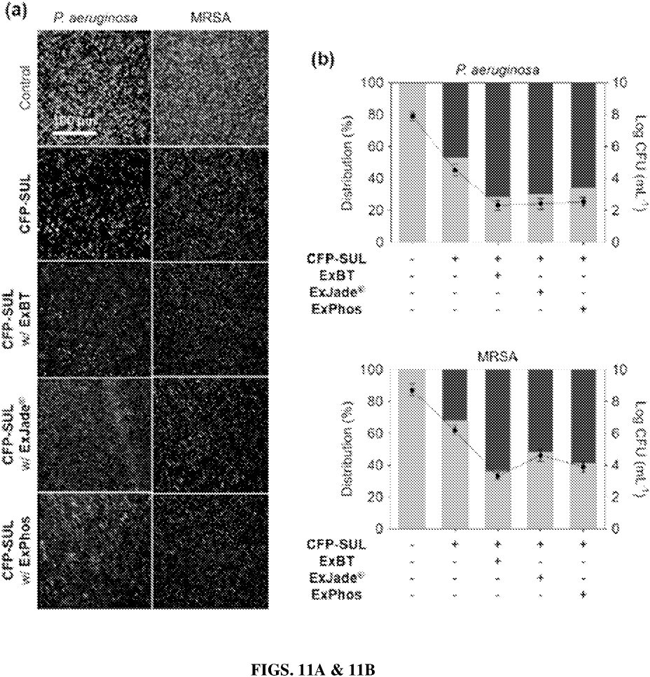

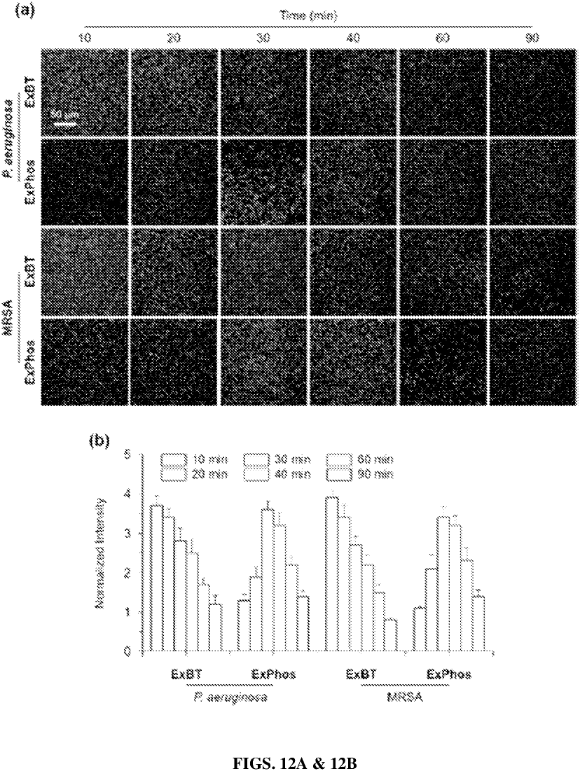

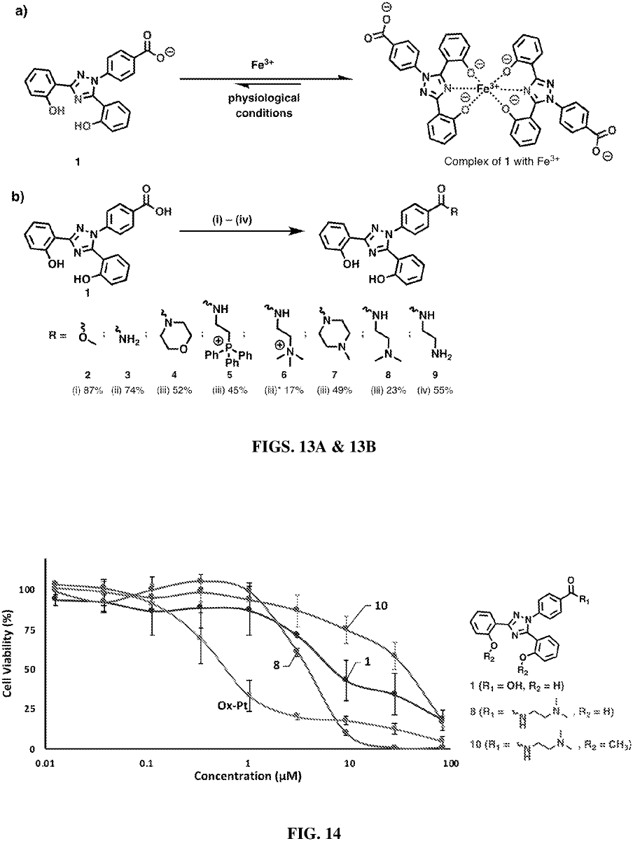

The Fe(III) chelation properties of ExBT were then evaluated. Evidence of interaction with Fe(III) first came from UV-visible spectral studies. These initial experiments were performed in an organic solvent (DMSO) as this facilities rapid Fe(III) chelation. As seen previously for deferasirox, the addition of increasing concentrations of Fe(III) (as the FeCl₃ salt) to both ExBT (15 µM) and deferasirox (15 µM) resulted in an increase in their respective UV-vis absorption intensities, along with a color change from clear to reddish/purple (Steinhauser et al., 2004). No change in UV-vis absorption/color was seen when Fe(III) was added to ExBT-OMe. From this information, it may be concluded that a free phenol is needed to chelate Fe(III) (Steinhauser et al., 2004). A concentration-dependent quenching of the ExBT fluorescence was also seen (FIG. 10A), as would be expected for Fe(III) chelation (paramagnetic metal fluorescence quenching) (Yang et al., 2015).

Recently, protected versions of known metal chelators (“prochelators”) have been explored in an effort to avoid premature metal chelation (Wang et al., 2016). The bis-phosphate ester ExPhos was thus prepared as a phosphatase-responsive fluorescent pro-chelator that would permit detection of a disease-based biomarker (i.e., alkaline phosphatase, ALP) (Zhang et al., 2020; McCullough and Barrios, 2020; Haarhaus et al., 2017; Gwynne et al., 2019). As true for the protected system, ExBT-OMe, minimal Fe(III) quenching or change in the UV-Vis absorption was seen for ExPhos (FIG. 10C). However, in the presence of ALP a dose dependent change in the fluorescence emission was observed (0-64 U), with a final fluorescence emission profile analogous to that of ExBT being seen (FIG. 10E). Timedependent changes in fluorescence emission intensity were also seen. The limit of detection (LoD) was calculated to be 0.016 75 U (measurements performed after 5 min of incubation). The ratiometric change in the fluorescence emission was ascribed to the gradual and stepwise (bis-phosphate ester to monophosphate ester to bisphenol) dephosphorylation of ExPhos (as confirmed by LCMS analyses). This sequential conversion leads to a rapid decrease in the blue emission and a slow increase in the green emission. ExPhos solutions exposed to ALP (64 U ALP) were subsequently treated with increasing concentrations of Fe(III) (as FeCl₃). Quenching of the fluorescence intensity was observed (FIG. 10D).

Both Fe(III) concentration and ALP activity are key to the growth of pathogenic bacteria including biofilm formation and development (Post et al., 2019; Huang et al., 1998; Danikowsk et al., 2018; Katsipis et al., 2021; O’May et al., 2009; Rao and Torriani, 1990). Biofilms are complex bacterial communities enclosed by extracellular polymeric substances (EPS), which provide protection against antibiotics (Hall and Mah, 2017; Stephens et al., 2020). In addition, biofilms can rapidly adapt to their environments (Hall and Mah, 2017). These characteristics result in hard-to-treat infections, promote antibiotic resistance, and lead to patient complications (Sharma et al., 2019). This provides an incentive to develop fluorescent tools to image biofilms and visualize biomarkers associated with their formation and survival.

To date, several AIEgens have been reported for bacteria-based imaging applications (Feng et al., 2021). For instance, Tang and coworkers reported an AIEgen that can discriminate between Gram-positive bacteria, Gram-negative bacteria, and fungi (Zhou et al., 2020), while Liu and co-workers detailed a dual-functional TPE-based antibiotic AIEgen that allows for the intracellular imaging of bacteria in living host cells (Hu et al., 2020). However, little attention has been paid to imaging biofilms. Thus, the ExBT and ExPhos compounds were evaluated for their diagnostic and antibiofilm potential.

Biofilm experiments were carried out on glass slides with both Pseudomonas aeruginosa and MRSA. The therapeutic performance of the broad-spectrum antibiotic cefoperazonesulbactam (CFP-SUL) (Sheu et al., 2020) was evaluated on its own and in combination with deferasirox, ExBT, and ExPhos using the live/dead biofilm caption viability assay (propidium iodide (PI): dead, red; Syto9: live, green). As shown in the processed 3D images (FIG. 11), the use of just CFP-SUL led to a partial antibiofilm effect that was enhanced in the presence of ExBT, ExPhos, or deferasirox.

Subsequent confocal fluorescence imaging experiments were performed using both P. aeruginosa and MRSA biofilms with ExBT and ExPhos (FIG. 12). The green fluorescence emission seen in FIG. 11 and FIG. 12 arises from Syto9 and ExBT, respectively. Thus, to avoid interference, the development of fluorescent deferasirox derivatives with long-wavelength excitation/emission profiles would be desirable for biological applications. In both the P. aeruginosa and MRSA biofilms, ExBT gives rise to a strong fluorescence emission, which gradually diminished with time. In contrast, ExPhos displayed an initial weak fluorescence emission, whose intensity gradually increased but then decreased at later times. A phosphatase-dependent fluorescence turn-on response was observed for MRSA (ATCC 43300). These evolving spectral features are interpreted in terms of direct Fe(III) chelation vs initial dephosphorylation followed by Fe(III) chelation in the case of ExBT and ExPhos, respectively.

As detailed above, analogues of the FDA-approved iron chelator deferasirox have been prepared that display unique AIE, charge transfer, and through-space conjugation characteristics in aqueous media. This platform allowed development of a stimulus-responsive fluorescent pro-chelator, ExPhos, which was designed to prevent premature Fe(III) chelation while enabling the detection of the bacteria-based biomarker alkaline phosphatase. Thus, these modified deferasirox derivatives can be used for cellular imaging applications including the detection of disease-based biomarkers.

### Example 4: In Vitro Studies of Deferasirox Derivatives as Potential Organelle-Targeting Traceable Anti-Cancer Therapeutics

### I. Introduction

A particularly appealing target for optimization is deferasirox (1, FIG. 13), an FDA approved iron sequestration agent with a well-established pharmacological profile. It has been shown that under physiological conditions, 1 binds Fe³⁺ selectively over other biorelevant metal cations to form a 2:1 ligand-to-cation complex stabilised via the ONO donor set (FIG. 13A) (Steinhauser et al., 2004). Initial studies of deferasirox revealed promising anticancer activity in vitro and in vivo against inter alia AML (Callens et al., 2010), triple negative breast cancer (Tury et al., 2018), and lung cancer (Lui et al., 2013; Loza-Rosas et al., 2017). Moreover, deferasirox 1 can be prepared on large scale from a range of commercially accessible building blocks, a feature that could allow for rapid synthetic modification. Within this context, preparative derivatisation of the terminal carboxylic acid in 1 is particularly attractive, since this functionality imparts an overall negative charge on the ligand at physiological pH (FIG. 13). This precludes effective passage through lipid cell membranes and furthermore renders the ligand non-fluorescent in water (Huang et al., 2006; Manzoori et al., 2011). The strategic functionalization of 1 provides access to chelators that show fluorescent behaviour in aqueous media (Sedgwick et al., 2020).

### II. Results

The antiproliferative activity of several deferasirox derivatives has been examined using the A549 human lung cancer cell line and as detailed below identified derivative 8 as having an enhanced therapeutic activity as compared to 1. The fluorescent nature of 8 allowed its subcellular localisation in the lysosome to be followed by fluorescent microscopy.

The deferasirox derivatives of the present study were prepared by modifying the terminal carboxylic acid moiety in 1 so as to introduce neutral, cationic and protonatable amine-containing side chains. This gave a set of compounds that were designed to allow the relationship between structure and cellular uptake and organelle targeting to be tested. First, guided by the premise that they would benefit from improved passive diffusion through lipid membrane and thus faster internalization with respect to 1, several derivatives with neutral sidechains were prepared (compounds 2-4, FIG. 13B). Next, derivatives were synthesized containing hydrophilic amine and ammonium side chains (6-9, FIG. 13B) as these functionalities have been reported to promote preferential localization in the lysosome (Corcé et al., 2012; Graff et al., 2001). Finally, one derivative (5, FIG. 13B) was prepared bearing a cationic triphenylphosphonium moiety, a hydrophobic subunit reported to drive localisation toward the mitochondria (Zielonka et al., 2017; Alta et al., 2017). Previously described derivatization of the carboxylic acid moiety of 1 include reports by Salehi et al. who employed conventional amide coupling methods in the synthesis of a glycine- and a phenylalanine methyl ester derivatives (Salehi et al., 2016), as well as by Rouge et al. who tethered a calix[4]arene to 1 via an ethylene glycol linker (Rouge et al., 2012). These previously reported systems differ from targets 2-9, which were thus expected to sample different structure-activity space that had not hitherto been explored in the context of probing the anticancer activity of deferasirox derivatives.

All compounds prepared herein were characterized by ¹H- and ¹³C-NMR spectroscopy, as well as by high resolution mass spectrometry. Deferasirox 1 was prepared in two steps via a modified procedure that had been reported previously (Steinhauser et al., 2004).

TABLE 1

IC₅₀ and Hill slope (HS) values for 1-10 against the A549 human lung cancer cell line after 72 h and 24 h incubation. Experiments were performed in triplicate.

| Compound | IC₅₀ (72 h) | HS (72 h) | IC₅₀ (24 h) | HS (24 h) |
|---|---|---|---|---|
| Ox-Pt | 0.5 ± 0.1 µM | 1.2 ± 0.2 | >50 µM | N/A |
| 1 | 8.5 ± 2.0 µM | 1.0 ± 0.2 | >50 µM | N/A |
| 2 | 2.5 ± 0.7 µM | 0.9 ± 0.2 | 21.0 ± 3.0 µM | 1.3 ± 0.2 |
| 3 | 8.3 ± 2.2 µM | 0.8 ± 0.2 | >50 µM | N/A |
| 4 | 3.6 ± 1.6 µM | 0.6 ± 0.2 | >50 µM | N/A |
| 5 | 6.0 ± 0.6 µM | 1.9 ± 0.3 | 29.0 ± 1.4 µM | 2.9 ± 0.6 |
| 6 | >50 µM | N/A | >50 µM | N/A |
| 7 | 3.8 ± 0.5 µM | 1.0 ± 0.1 | 23.3 ± 1.5 µM | 1.8 ± 0.2 |
| 8 | 3.7 ± 0.3 µM | 2.7 ± 0.6 | 12.3 ± 1.3 µM | 3.5 ± 1.0 |
| 9 | 7.7 ± 0.8 µM | 1.7 ± 0.3 | 12.6 ± 0.8 µM | 2.7 ± 0.4 |
| 10 | >50 µM | N/A | >50 µM | N/A |

The new derivatives were prepared via Fischer esterification (2), microwave assisted amidation adapted from a protocol developed by Khalafi-Nezhad et al. (3) (Khalafi-Nezhad et al., 2003), and carbodiimide mediated peptide coupling with N-hydroxysuccinimide catalysis (4-10), respectively.

The antiproliferative activity of compounds 1-10 was evaluated in A549 lung cancer cells, a cell line with a well- established sensitivity to iron imbalance (Loza-Rosas et al., 2017; Ohara et al., 2013). For each compound, cellular proliferation profiles were produced via a standard MTT (3-(4,5-dimethylthiazol-2-yl)-2,5-diphenyltetrazolium bromide) assay for exposure times of 72 h and 24 h, respectively. From these proliferation profiles, averaged IC₅₀ values and Hill slope (HS) parameters were determined via linear regression analysis. The results are summarised in Table 1.

IC₅₀ values represent a commonly reported metric of toxicity, while HS parameters provide insight into the shape of the proliferation profile, in particular, the steepness of the dose-response curve. HS parameters have attracted interest in recent years as providing a means for comparing the activity of compounds across different cell lines that is more consistent than the IC₅₀ value. Non-plateauing, steep dose-response curves are considered favourable for chemotherapeutics because small, clinically achievable increases in the concentration of a drug above its IC₅₀ value can translate into a disproportionally larger fractional killing of cancer cells (Fallahi-Sichani et al., 2013; Jenkins, 2013).

As positive control oxaliplatin (Ox-Pt), a platinum based antineoplastic agent that is in current clinical use as chemotherapeutic and has a well-established activity profile in A549 cells, was included (Zhang et al., 2019; Raveendran et al., 2016). However, in this cell line, Ox-Pt displays a rather shallow dose-response curve and prolonged treatments with high concentrations of Ox-Pt are required to achieve an effective response. Ox-Pt is recognized for a number of unwanted side effects, including neuropathy (Zhang et al., 2019; Raez and Santos, 2010). While the actual clinical determinants are likely multi-factorial, agents with more favorable HS parameters and improved IC₅₀ values may be free of some of these drawbacks.

The IC₅₀ values of 1 and Ox-Pt are in agreement with previous literature reports for an exposure time of 72 h (Loza-Rosas et al., 2017; Zhang et al., 2019; Raveendran et al., 2016). At this incubation time, the new derivatives of this study showed activities that were either improved relative to 1 or similar. The exception was 6, which proved inactive. Notably, the methyl ester 2 (IC₅₀ = 1.6 µM), morpholine amide 4 (IC₅₀ = 2.6 µM) the cationic triphenylphosphonium bearing derivative 5 (IC₅₀ = 6.0 µM) and the free amines 8 (IC₅₀ = 3.7 µM) and 9 (IC₅₀ = 4.3 µM) were found to be 2-3 times more cytotoxic than the parent chelator 1 (IC₅₀ = 8.5 µM). Reducing the exposure time to 24 h decreased the apparent activity of the charge neutral derivatives 2-4, as well as Ox-Pt by over an order of magnitude. This finding suggests that several cell cycles are required for these compounds to exert a cytotoxic effect. Interestingly, the activity of the derivatives with organelle targeting functionalities, the triphenylphosphonium salt 5, as well as the amines 7, 8 and 9, were less impacted by shorter exposure times with 8 (IC₅₀ = 12.3 µM) and 9 (IC₅₀ = 12.6 µM) exhibiting the highest activity under these conditions. The organelle-targeting derivatives 5, 8, and 9 produced the highest HS parameters after both 72 h and 24 h exposures, while 1, Ox-Pt, and the derivatives with neutral side chains (i.e., 2-4) produced rather shallow dose-response curves (Table 1).

The combination of favorable cytotoxicity and HS seen at 72 h in the case of 8 led to the preparation of its diether analogue 10 via methylation of the phenol moieties. This derivative was expected to display a reduced metal binding affinity thus serving as a negative control for 8. In fact, derivative 10 exerted no appreciable antiproliferative activity against A549 cells under conditions identical to those used to test compounds 1-9. Additionally, when cells were supplemented with 50 µM FeCl₃, both 1 and 8 no longer produced any observable cytotoxicity after 72 h of exposure. This finding is taken as evidence that intracellular iron chelation plays a key role in mediating their in vitro antiproliferative activity.

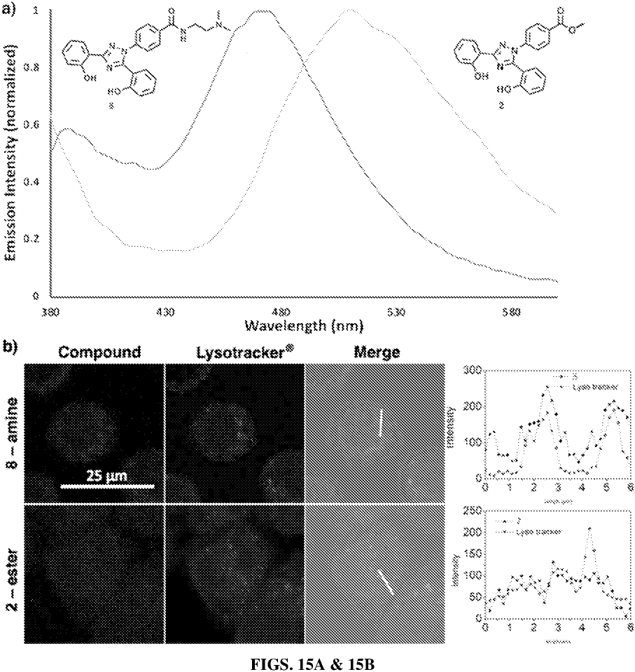

In contrast to deferasirox 1, derivatives 8 and 2 at concentrations of 50 µM give rise to distinct fluorescence emission bands centred 480 nm and 510 nm, respectively, in phosphate buffered saline (PBS, pH = 7.2) (cf. FIG. 15). This allowed their subcellular location to be explored. As confirmed by comparisons with Lysotracker® red, derivative 8 was found to localise in the lysosome as determined by fluorescent cell microscopy using HeLa cells. In contrast, no discernible organelle targeting was seen in the case of 2, a chelator that lacks a recognized organelle targeting unit. These findings suggest that the enhanced therapeutic efficacy seen in the case of 8 is due, at least in part, to its intracellular localization.

The eight new derivatives of deferasirox, including examples with neutral, cationic and amine-containing side chains, were evaluated for their antiproliferative activity in A549 cells after incubation times of 24 h and 72 h. The derivatives that contained organelle targeting moieties, such as 8, were found to exert notable cytotoxicity after 24 h exposure time and also showed steeper dose-response curves with respect to the parent chelator 1, as well as oxaliplatin used as a positive control. Derivative 8, as well as the majority of the new compounds reported here, proved fluorescent in aqueous media, allowing their subcellular localisation to be tracked inside live cells. It was found that the chelator 8, but not the control system 2 lacking a localizing functionality, localized well to the lysosome. The ability to produce an antiproliferative response as well as providing for fluorescence-based tracking, are considered attractive features of the present systems and serve to underscore the versatility of the deferasirox platform in terms of potential iron chelation-based approaches to anticancer drug discovery. More broadly, the present results highlight the benefits that can accrue by optimizing the drug-like properties and targeting features of chelators displaying therapeutic potential.

All of the compounds, formulations, and methods disclosed and claimed herein can be made and executed without undue experimentation in light of the present disclosure. While the compounds, formulations, and methods of this invention have been described in terms of preferred embodiments, it will be apparent to those of skill in the art that variations may be applied to the compounds, formulations, and methods, as well as in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
Alta et al., BioMetals, 30, 709-718, 2017.
Anderson, Practical Process Research & Development - A Guide for Organic Chemists, 2^{nd} ed., Academic Press, New York, 2012.
Bedford et al., J. Clin. Pharmacol., 53, 885-891, 2013.
Bogdan et al., Trends Biochem. Sci., 41, 274-286, 2016.
Braibant et al., FEMS Microbiol. Lett., 195(2):121-126, 2001.
Callens et al., J. Exp. Med., 2010, 207, 731-750.
Cassat and Skaar, Cell Host Microbe, 13(5), 510-520, 2013.
Chen et al., Mater. Horiz., 6(3), 428-433, 2019.
Corcé et al., Bioconjug. Chem., 23, 1952-1968, 2012.
Dahm et al., Chem. Asian J., 10:2368-2379, 2015.
Danikowsk et al., Curr. Microbiol., 75(9), 1226-1230, 2018.
Das et al., Phys. Chem. Chem. Phys., 21(28):15669-15677, 2019.
Dou et al., Sci. Bull., 64(24), 1902-1909, 2019.
Duarte et al., Cell Stem Cell, 22, 64-77.e6, 2018.
Fallahi-Sichani et al., Nat. Chem. Biol., 9, 708-714, 2013.
Feng et al., Mater. Chem. Front., DOI: 10.1039/D0QM00753F, 2021.
Filipowicz et al., Phytother. Res., 17(3):227-231, 2003.
Fryknäs et al., Sci. Rep., 6, 1-11, 2016.
Fukushima et al., Anticancer Res., 31, 1741-1744, 2011.
Gaur et al., Inorganics, 2018, DOI:10.3390/inorganics6040126.
Graff et al., Cancer Res., 61, 2138-2144, 2001.
Gwynne et al., Front. Chem., 7, 2019, DOI: 10.3389/fchem.2019.00255.
Haarhaus et al., Nat. Rev. Nephrol., 13(7), 429- 442, 2017.
Hall and Mah, FEMS microbiol. Rev., 41(3), 276-301, 2017.
Handbook of Pharmaceutical Salts: Properties, and Use, Stahl and Wermuth Eds., Verlag Helvetica Chimica Acta, 2002.
Hao and Chen, Dyes Pigm., 129:186-190, 2016.
He et al., Org. Chem. Front., 3(9), 1091-1095, 2016.
Heath et al., Nutrients, 5, 2836-2859, 2013.
Hennigar and McClung, Am. J. Lifestyle Med., 10(3):170-173, 2016.
Hong et al., Chem. Comm., 29:4332-4353, 2009
Hu et al., Angew. Chem., Int. Ed., 59(24), 9288-9292, 2020.
Huang et al., Appl. Environ. Microbiol., 64(4), 1526-1531, 1998.
Huang et al., Pharm. Res., 23, 280-290, 2006.
Jenkins, Nat. Chem. Biol., 9, 669-670, 2013.
Jung et al., Int. J. Mol. Sci., 20(2), 273, 2019.
Jung et al., Int. J. Mol. Sci., 20, 1-18, 2019.
Katsipis et al., Appl. Microbiol. Biotechnol., 105, 147, 2021.
Katsura et al., Cancers (Basel)., 11, 1-16, 2019.
Khalafi-Nezhad et al., Tetrahedron Lett., 44, 7325-7328, 2003.
Kielar et al., Inorg. Chim. Acta, 393, 294-303, 2012.
Kim et al., J. Photochem. Photobio. A Chem., 191(1):19-24, 2007.
Kriakov et al., J. Bacteriol., 185(16):4983-4991, 2003.
Lin et al., PLoS One, 7(3), e34388, 2012.
Liu et al., Angew. Chem., Int. Ed., 56(39), 11788-11792, 2017.
Lou et al., J. Phys. Chem. C, 122(1):185-193, 2018.
Loza-Rosas et al., Inorg. Chem., 56, 7788-7802, 2017.
Lui et al., Mol. Pharmacol., 83, 179-190, 2013.
Mai et al., Nat. Chem., 9, 1025-1033, 2017.
Manzoori et al., Luminescence, 26, 244-250, 2011.
McCullough and Barrios, Curr. Opin. Chem. Biol., 57, 34-40, 2020.
Miller et al., Acc. Chem. Res., 26(5):241-249, 1993.
Mislin and Schalk, Metallomics, 6(3):408-420, 2014.
Mody et al., Invest. New Drugs, 37, 684-692, 2019.
Moon et al., J. Med. Microbiol., 62, 1307-1316, 2003.
Moreau-Marquis et al., Am. J. Respir. Cell Mol. Biol., 41(3), 305-313, 2009.
Moukalled et al., J. Hematol. Infect. Dis., 10, e2018066, 2017.
Mulani et al., Front. Microbiol., 10, 2019.
Negash et al., Molecules, 24(18), 2019.
O’May et al., J. Med. Microbiol. 2009, 58 (6), 765-773.
Oh et al., Front. Microbiol. 2018, 9, DOI: 10.3389/fmicb.2018.01204.
Ohara et al., Int. J. Cancer, 132, 2705-2713, 2013.
Pannipara et al., Spectrochim. Acta A., 136:1893-1902, 2015.
Pathak and Dhar, J. Am. Chem. Soc., 137:8324-8327.
Peeters et al., Infect. Drug Resist., 12:329-343, 2019.
Peng et al., Anal. Chem., 89(5), 3162- 3168, 2017.
Peng et al., J. Am. Chem. Soc., 137(45):14349-14357, 2015.
Post et al., MedChemComm, 10(4), 505-512, 2019.
Pramanik et al., RSC Adv., 5:101959-101964, 2015.
Puri et al., Agents Chemother., 63(4), 2019, DOI: 10.1128/AAC.02152-18.
Qian et al., Chem., 3(1):56-91, 2017.
Qu et al., Chem. Commun., 48(38):4600-4602, 2012.
Raez and Santos, Clin. Lung Cancer, 11, 18-24, 2010.
Rao and Torriani, Mol. Microbiol., 4(7), 1083-1090, 1990.
Raveendran et al., Chem. Sci., 7, 2381-2391, 2016.
Reagan-Shaw et al., FASEB J., 22(3):659-661, 2008.
Rouge et al., Chempluschem, 77, 1001-1016, 2012.
Rugina, J. Crit. Care Med., 4(2):47-49, 2018.
Saeki et al., World J. Gastroenterol., 22, 8967-8977, 2016.
Sahu et al., Phys. Chem. Chem. Phys., 20(42):27131-27139, 2018.
Sajid et al., Annu. Rev. Microbiology, 69:527-547, 2015.
Salehi et al., Eur. J. Pharmacol., 781, 209-217, 2016.
Sasaki et al., J. Mater. Chem. C, 4(14), 2731-2743, 2016.
Sedgwick et al., Chem. Soc. Rev., 47(23):8842-8880, 2018.
Sedgwick et al., ChemRxiv. 2020, Preprint. https://doi.org/10.26434/chemrxiv.12040518.v1
Sedgwick et al., J. Am. Chem. Soc., 140(43):14267-14271, 2018.
Sharma et al., Antimicrob. Resist. Infect. Control, 8, 2019, DOI: 1 0.1186/s 13756-019-0533-3.
Shen et al., J. Phys. Chem. Lett., 10(11), 2648-2656, 2019.
Sheu et al., Antibiotics, 9(2), 77, 2020.
Shirley and Plosker, Drugs 2014, 74 (9), 1017-1027.
Sivalingam et al., Chempluschem, 84(4):392-402, 2019.
Smith, March’s Advanced Organic Chemistry: Reactions, Mechanisms, and Structure, 7^{th} Ed., Wiley, 2013.
Steinbrueck et al., Chem. Soc. Rev., 49, 3726-3747, 2020.
Steinhauser et al., Eur. J. Inorg. Chem., 21:4177-4192, 2004.
Stephens et al., Future Med. Chem., 12(22), 2035-2065, 2020.
Tang et al., J. Phys. Chem. Lett., 2(24):3063-3068, 2011.
Theerasilp et al., RSC Adv., 7, 11158-11169, 2017.
Thompson et al., Antimicrob. Agents Chemother., 56(10):5419-5421, 2012.
Tian et al., Coord. Chem. Rev., 427, 213577, 2021.
Torti and Torti, Nat. Rev. Cancer, 13, 342-355, 2013.
Tury et al., J. Pathol., 246, 103-114, 2018.
Vollmer and Rettig, Photobiology α-Chemistry, 95(2):143-155, 1996.
von Bubnoff, Cell, 127(5):867-869, 2006.
Wang et al., Acc. Chem. Res., 49(11), 2468- 2477, 2016.
Wang et al., Mater. Today, 18(7), 365-377, 2015.
Wright et al., Angew Chem. Int. Ed., 53(34):8840-8869, 2014.
Wu et al., Chem. Commum., 54(80):11336-11339, 2018.
Yang et al., Chem. Commun. 51(47), 9616-9619, 2005.
Yang et al., Drugs, 67(15), 2211-2230, 2007.
Yang et al., Mater. Chem. Front., 2(5), 861- 890, 2018.
Yoshihara et al., J. Phys. Chem. A, 109(8):1497-1509, 2005.
Yoshihara et al., Photochem. Photobiol. Sci., 2(3):342-353, 2003.
Yu et al., Curr. Med. Chem., 19, 2689-2702, 2012.
Yu et al., J. Med. Chem., 52, 5271-5294, 2009.
Zhang et al., Anal. Chem., 92(7), 5185-5190, 2020.
Zhang et al., Angew. Chem., Int. Ed., 58(26), 8773-8778, 2019.
Zhang et al., J. Med. Chem., 62, 4543-4554, 2019.
Zhao et al., Phys. Chem. Chem. Phys., 17(18):11990-11999, 2016.
Zhou et al., Chem. Sci., 11(18), 4730- 4740, 2020.
Zielonka et al., Chem. Rev., 117, 10043-10120, 2017.

What is claimed is:

1. A compound of the formula: 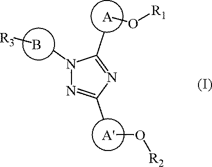
wherein:
A and A′ are arenediyl_{(C≤12)}, heteroarenediyl_{(C≤12)}, or a substituted version thereof;
B is —X₁—Y₁—, wherein:
X₁ is a covalent bond, alkanediyl_{(C≤8)}, substituted alkanediyl_{(C≤8)}, arenediyl_{(C≤12)}, or substituted arenediyl_{(C≤12)}; and
Y₁ is absent, -NR₄-, —C(O)—, —C(O)O—, -C(O)NR₄-, arenediyl_{(C≤12)}, heteroarenediyl_{(C≤12)}, heterocycloalkanediyl_{(C≤12)}, or a substituted version wherein:
R₄ is hydrogen, alkyl_{(C≤6)}, substituted alkyl_{(C≤6)}, or a monovalent amine protecting group;
R₁ and R₂ are each independently a moiety cleavable to hydrogen; and
R₃ is hydrogen or alkyl_{(C≤12)}, aryl_{(C≤12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
or a pharmaceutically acceptable salt thereof.

2. The compound of claim 1 further defined as: 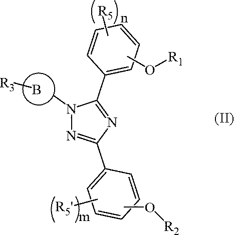
wherein:
B is —X₁—Y₂—, wherein:
X₁ is a covalent bond, alkanediyl_{(C≤8)}, substituted alkanediyl_{(C≤8)}, arenediyl_{(C≤12)}, or substituted arenediyl_{(C≤12)}; and
Y₁ is absent, -NR₄-, —C(O)—, —C(O)O—, -C(O)NR₄-, arenediyl_{(C≤12)}, heteroarenediyl_{(C≤12)}, heterocycloalkanediyl_{(C≤12)}, or a substituted version wherein:
R₄ is hydrogen, alkyl_{(C≤6)}, substituted alkyl_{(C≤6)}, or a monovalent amine protecting group;
R₁ and R₂ are each independently a moiety cleavable to hydrogen;
R₃ is hydrogen or alkyl_{(C≤12)}, aryl_{(C≤12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
R₅ and R₅′ are each independently hydrogen, halo, or hydroxy; and
m and n are each 1, 2, or 3;
or a pharmaceutically acceptable salt thereof.

3. The compound according to either claim 1 or claim 2 further defined as: 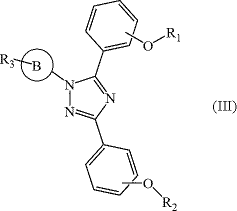
wherein:
B is —X₁—Y₁—, wherein:
X₁ is a covalent bond, alkanediyl_{(C≤8)}, substituted alkanediyl_{(C≤8)}, arenediyl_{(C≤12)}, or substituted arenediyl_{(C≤12)}; and
Y₁ is absent, -NR₄-, —C(O)—, —C(O)O—, -C(O)NR₄-, arenediyl_{(C≤12)}, heteroarenediyl_{(C≤12)}, heterocycloalkanediyl_{(C≤12)}, heterocycloalkalkyl_{(C≤12)}, or a substituted version wherein:
R₄ is hydrogen, alkyl_{(C≤6)}, substituted alkyl_{(C≤6)}, or a monovalent amine protecting group;
R₁ and R₂ are each independently a moiety cleavable to hydrogen; and
R₃ is hydrogen or alkyl_{(C≤12)}, aryl_{(C≤12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
or a pharmaceutically acceptable salt thereof.

4. The compound according to any one of claims 1-3 further defined as: 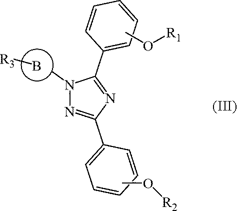
wherein:
B is —X₁—Y₁—, wherein:
X₁ is arenediyl_{(C≤12)} or substituted arenediyl_{(C≤12)}; and
Y₁ is absent, —C(O)—, or —C(O)O—;
R₁ and R₂ are each independently a moiety cleavable to hydrogen; and
R₃ is hydrogen or alkyl_{(C≤12)}, aryl_{(C≤12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
or a pharmaceutically acceptable salt thereof.

5. The compound of claim 1, wherein A is arenediyl_{(C≤12)} or substituted arenediyl_{(C≤12)}.

6. The compound of claim 5, wherein A is arenediyl_{(C≤12)}.

7. The compound of claim 6, wherein A is benzenediyl.

8. The compound according to any one of claim 1 and 5-7, wherein A′ is arenediyl_{(C≤12)} or substituted arenediyl_{(C≤12)}.

9. The compound of claim 8, wherein A′ is arenediyl_{(C≤12)}.

10. The compound of claim 6, wherein A′ is benzenediyl.

11. The compound according to any one of claims 2 and 5-10, wherein R₅ is hydrogen.

12. The compound according to any one of claims 2 and 5-10, wherein R₅ is halo.

13. The compound according to any one of claims 2 and 5-10, wherein R₅ is hydroxy.

14. The compound according to any one of claims 2 and 5-13, wherein R₅′ is hydrogen.

15. The compound according to any one of claims 2 and 5-13, wherein R₅′ is halo.

16. The compound according to any one of claims 2 and 5-13, wherein R₅′ is hydroxy.

17. The compound according to any one of claims 2 and 5-16, wherein m is 1 or 2.

18. The compound according to any one of claims 2 and 5-17, wherein n is 1 or 2.

19. The compound according to any one of 1-18, wherein X₁ is a covalent bond.

20. The compound according to any one of 1-18, wherein X₁ is alkanediyl_{(C≤8)} or substituted alkanediyl_{(C≤8)}.

21. The compound of claim 20, wherein X₁ is alkanediyl_{(C≤8)}.

22. The compound of claim 21, wherein X₁ is methylene or ethylene.

23. The compound according to any one of claims 1-18, wherein X₁ is arenediyl_{(C<12)} or substituted arenediyl_{(C≤12)}.

24. The compound of claim 23, wherein X₁ is arenediyl_{(C≤12)}.

25. The compound of claim 24, wherein X₁ is benzenediyl.

26. The compound according to any one of claims 1-25, wherein Y₁ is absent.

27. The compound according to any one of claims 1-25, wherein Y₁ is —C(O)—.

28. The compound according to any one of claims 1-25, wherein Y₁ is —C(O)O—.

29. The compound according to any one of claims 1-3 and 5-25, wherein Y₁ is -C(O)NR₄-.

30. The compound of claim 29, wherein R₄ is hydrogen.

31. The compound of claim 29, wherein R₄ is alkyl_{(C≤6)} or substituted alkyl_{(C≤6)}.

32. The compound of claim 31, wherein R₄ is alkyl_{(C≤6)}.

33. The compound of claim 32, wherein R₄ is methyl.

34. The compound of claim 33, wherein R₄ is substituted alkyl_{(C≤6)}.

35. The compound of claim 34, wherein R₄ is 2-hydroxyethyl.

36. The compound according to any one of claims 1-35, wherein R₃ is hydrogen.

37. The compound according to any one of claims 1-35, wherein R₃ is alkyl_{(C≤12)} or substituted alkyl_{(C≤12)}.

38. The compound of claim 37, wherein R₃ is alkyl_{(C≤12)}.

39. The compound of claim 38, wherein R₃ is methyl or ethyl.

40. The compound of claim 37, wherein R₃ is substituted alkyl_{(C≤12)}.

41. The compound of claim 40, wherein R₃ is 2-hydroxyethyl, 2-methoxyethyl, or 2,3-dihydroxyethyl.

42. The compound according to any one of claims 1-35, wherein R₃ is aryl_{(C≤12)} or substituted aryl_{(C≤12)}.

43. The compound of claim 42, wherein R₃ is aryl_{(C≤12)}.

44. The compound of claim 43, wherein R₃ is phenyl or napthyl.

45. The compound of claim 42, wherein R₃ is substituted aryl_{(C≤12)}.

46. The compound of claim 45, wherein R₃ is 4-nitrophenyl, 4-methoxyphenyl, or 4-nitrophenyl.

47. The compound according to any one of claims 1-35, wherein R₃ is aralkyl_{(C≤12)} or substituted aralkyl_{(C≤12)}.

48. The compound of claim 47, wherein R₃ is aralkyl_{(C≤12)}.

49. The compound of claim 48, wherein R₃ is benzyl.

50. The compound according to any one of claims 1-35, wherein R₃ is heteroaryl_{(C≤12)} or substituted heteroaryl_{(C≤12)}.

51. The compound of claim 50, wherein R₃ is heteroaryl_{(C≤12)}.

52. The compound of claim 43, wherein R₃ is pyridinyl or benzothiazolyl.

53. The compound according to any one of claims 1-35, wherein R₃ is heterocycloalkyl_{(C≤12)} or substituted heterocycloalkyl_{(C≤12)}.

54. The compound of claim 53, wherein R₃ is heterocycloalkyl_{(C≤12)}.

55. The compound of claim 54, wherein R₃ is N-methylpiperazinyl, morpholinyl, or pyrrolidinyl.

56. The compound according to any one of claims 1-35, wherein R₃ is alkoxy_{(C≤12)} or substituted alkoxy_{(C≤12)}.

57. The compound of claim 56, wherein R₃ is alkoxy_{(C≤12)}.

58. The compound of claim 57, wherein R₃ is methoxy or ethoxy.

59. The compound according to any one of claims 1-35, wherein R₃ is heterocycloalkalkyl_{(C≤12)} or substituted heterocycloalkalkyl_{(C≤12)}.

60. The compound of claim 59, wherein R₃ is heterocycloalkalkyl_{(C≤12)}.

61. The compound of claim 60, wherein R₃ is N′-methylpiperazinyl-N-2-ethyl.

62. The compound according to any one of claims 1-35, wherein R₃ is alkylamino_{(C≤12)} or substituted alkylamino_{(C≤12)}.

63. The compound of claim 62, wherein R₃ is alkylamino_{(C≤12)}.

64. The compound of claim 63, wherein R₃ is methylamino or ethylamino.

65. The compound of claim 62, wherein R₃ is substituted alkylamino_{(C≤12)}.

66. The compound of claim 65, wherein R₃ is 2-ethoxyethyl or 1-hydroxymethyl-2-hydroxyethyl.

67. The compound according to any one of claims 1-35, wherein R₃ is dialkylamino_{(C≤12)} or substituted dialkylamino_{(C≤12)}.

68. The compound of claim 67, wherein R₃ is dialkylamino_{(C≤12)}.

69. The compound of claim 68, wherein R₃ is dimethylamino or diethylamino.

70. The compound of claim 67, wherein R₃ is substituted dialkylamino_{(C≤12)}.

71. The compound of claim 70, wherein R₃ is N,N-di-(2-hydroxyethyl)amino.

72. The compound according to any one of claims 1-71, wherein R₁ or R₂ is moiety cleavable to hydrogen, wherein the moiety is cleaved by an enzyme that is substantially expressed by a microorganism.

73. The compound of claim 72, wherein the microorganism is a bacterium.

74. The compound of either claim 72 or claim 73, wherein the enzyme is preferentially expressed by a microorganism.

75. The compound of claim 74, wherein the enzyme is exclusively expressed by a microorganism.

76. The compound according to any one of claims 72-75, wherein the enzyme is an esterase, a phosphatase, or an enzyme which cleaves a bond to a sugar group.

77. The compound of claim 76, wherein the enzyme is a phosphatase or an enzyme which cleaves a bond to a sugar group.

78. The compound of either claim 76 or claim 77, wherein the enzyme which cleaves a bond to a sugar group is a glactosidase.

79. The compound according to any one of claims 1-71, wherein R₁ or R₂ is a moiety cleavable to hydrogen, wherein the moiety is cleaved in response to inflammation.

80. The compound of claim 79, wherein the moiety is cleaved by a molecule generated as a part of the inflammation response.

81. The compound of claim 80, wherein the molecule is a reactive oxygen species.

82. The compound of claim 81, wherein the reactive oxygen species is a superoxide or a peroxide.

83. The compound of claim 80, wherein the molecule is a chlorite.

84. The compound of claim 83, wherein the chlorite is hypochlorite.

85. The compound according to any one of claims 1-71, wherein R₁ or R₂ is a moiety cleavable to hydrogen, wherein the moiety is a therapeutic compound linked to the molecule by an ester, ether, hemiacetal, acetal, hemiketal, ketal, sulfonyl ether, sulfinyl ether, sulfonate ether, phosphate ester, boronate ester, or boronic acid.

86. The compound of claim 85, wherein the therapeutic compound is linked to the molecule by an ester group.

87. The compound of either claim 85 or claim 86, wherein the therapeutic compound is aspirin.

88. The compound according to any one of claims 72-87, wherein the moiety cleavable to hydrogen is further defined as a sugar or sugar derivative moiety or a functional group of the structure: 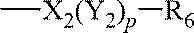
wherein:
X₂ is C, P, or S;
Y₂ is O or S;
p is 1 or 2; and
R₆ is hydrogen, amino, hydroxy, or alkyl_{(C≤12)}, aryl_{(C≤12)}, alkoxy_{(C≤12)}, alkylamino_{(C≤12)}, dialkylamino_{(C≤12)}, or a substituted version thereof, or a therapeutic compound.

89. The compound of claim 88, wherein X₂ is C.

90. The compound of claim 88, wherein X₂ is P.

91. The compound of claim 88, wherein X₂ is S.

92. The compound of either claim 88 or claim 89, wherein p is 1.

93. The compound according to any one of claims 88, 90, or 91, wherein p is 2.

94. The compound according to any one of claims 88-93, wherein Y₂ is O.

95. The compound according to any one of claims 88-93, wherein Y₂ is S.

96. The compound according to any one of claims 88-95, wherein R₆ is dialkylamino_{(C≤12)} or substituted dialkylamino_{(C≤12)}.

97. The compound of claim 96, wherein R₆ is dialkylamino_{(C≤12)}.

98. The compound of claim 97, wherein R₆ is dimethylamino.

99. The compound according to any one of claims 88-95, wherein R₆ is alkyl_{(C≤12)} or substituted alkyl_{(C≤12)}.

100. The compound of claim 99, wherein R₆ is alkyl_{(C≤12)}.

101. The compound of claim 100, wherein R₆ is methyl.

102. The compound of claim 99, wherein R₆ is substituted alkyl_{(C≤12)}.

103. The compound of claim 102, wherein R₆ is trifluoromethyl.

104. The compound according to any one of claims 88-95, wherein R₆ is aryl_{(C≤12)} or substituted aryl_{(C≤12)}.

105. The compound of claim 104, wherein R₆ is substituted aryl_{(C≤12)}.

106. The compound of claim 105, wherein R₆ is 2-acetoxyphenyl.

107. The compound according to any one of claims 88-95, wherein R₆ is hydroxy.

108. The compound of claim 88, wherein the moiety cleavable to hydrogen is a sugar or sugar derivative moiety.

109. The compound of claim 108, wherein the sugar moiety is linked by the anomeric carbon atom.

110. The compound of either claim 108 or claim 109, wherein the sugar moiety is a pentose or a hexose.

111. The compound of claim 110, wherein the sugar moiety is fructose, glucose, or galactose.

112. The compound of claim 111, wherein the sugar moiety is galactose.

113. The compound according to any one of claims 1-112, wherein R₁ and R₂ are the same group.

114. The compound according to any one of claims 1-112, wherein R₁ and R₂ are different groups.

115. The compound according to any one of claims 1-114, wherein the compound is further defined as: 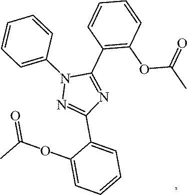 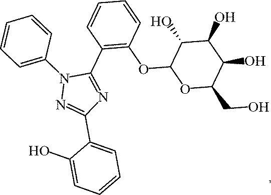 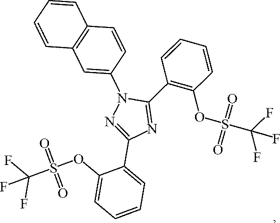 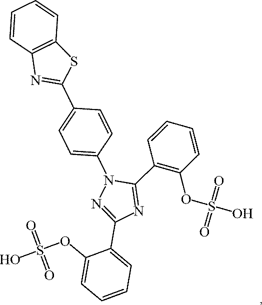 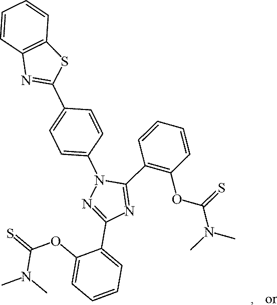 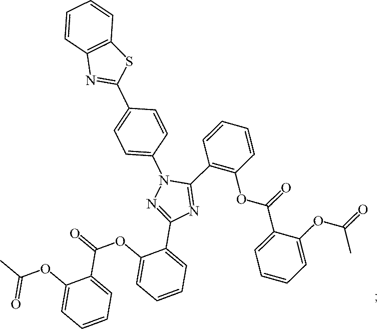
or a pharmaceutically acceptable salt thereof.

116. A pharmaceutical composition comprising:
(A) a compound according to any one of claims 1-115 or 177-215; and
(B) an excipient.

117. The pharmaceutical composition of claim 116, wherein the composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

118. The pharmaceutical composition of claim 117, wherein the composition is formulated for administration: topically, orally, or for injection.

119. The pharmaceutical composition according to any one of claims 116-118, wherein the pharmaceutical composition is formulated as a unit dose.

120. A method of treating a disease or disorder in a patient comprising administering to the patient in need thereof a therapeutically effective dose of a compound or pharmaceutical composition according to any one of claims 1-119 or 177-215.

121. The method of claim 120, wherein the disease or disorder is an infection of a microorganism.

122. The method of claim 121, wherein the microorganism is a bacterium.

123. The method of claim 121, wherein the microorganism is a virus.

124. The method of claim 120, wherein the disease or disorder is cancer.

125. The method of claim 124, wherein the cancer is the cancer is a carcinoma, sarcoma, lymphoma, leukemia, melanoma, mesothelioma, multiple myeloma, or seminoma.

126. The method of claim 124, wherein the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

127. The method according to any of claims 124-126, wherein the cancer is lung cancer.

128. The method according to any one of claims 120-127, wherein the method further comprises administering a second therapeutic agent.

129. The method of claim 128, wherein the second therapeutic agent is an antibiotic.

130. The method of claim 128, wherein the second therapeutic agent is an anti-viral.

131. The method according to any one of claims 120-130, wherein the patient is a mammal.

132. The method of claim 131, wherein the patient is a human.

133. A method of imaging an infection comprising contacting the microorganism with a compound or composition according to any one of claims 1-119 or 177-215 and detecting a change in signal.

134. The method of claim 133, wherein the signal is a change in fluorescence.

135. The method of either claim 133 or claim 134, wherein the signal is change in the adsorbance of visible or untraviolet light.

136. The method according to any one of claims 133-135, wherein the presence of the microorganism is indicated by the decrease in signal.

137. The method according to any one of claims 133-135, wherein the presence of the microorganism is a change in the λₘₐₓ.

138. The method according to any one of claims 133-137, wherein the microorganism is imaged in vivo.

139. The method according to any one of claims 133-137, wherein the microorganism is imaged in vitro.

140. The method according to any one of claims 133-137, wherein the microorganism is imaged ex vivo.

141. The method according to any one of claims 133-140, wherein the microorganism is a bacterium.

142. The method according to any one of claims 133-140, wherein the microorganism is a virus.

143. The method according to any one of claims 133-140, whereint the microorganism is a fungi.

144. The method according to any one of claims 133-143, wherein the microorganism is present as a biofilm.

145. A method of detecting a disease or disorder in a patient comprising contacting the patient with a compound or composition according to any one of claims 1-119 or 177-215, exposing the patient to a light source, and detecting a change in signal.

146. The method of claim 145, wherein the signal is a change in fluorescence.

147. The method of either claim 145 or claim 146, wherein the signal is change in the adsorbance of visible or untraviolet light.

148. The method according to any one of claims 145-147, wherein the disease or disorder is indicated by the decrease in signal.

149. The method according to any one of claims 145-147, wherein the disease or disorder is a change in the λₘₐₓ.

150. The method according to any one of claims 145-149, wherein the patient is imaged in vivo.

151. The mthod according to any one of claims 145-150, wherein the disease or disorder is an infection of a microorganism.

152. The method of claim 151, wherein the microorganism is a bacterium.

153. The method of claim 151, wherein the microorganism is a virus.

154. The method according to any one of claims 145-150, wherein the disease or disorder is cancer.

155. The method according to any one of claims 145-154, wherein the compound exhibits absorbance or fluorescence at two or more wavelengths.

156. The method of claim 155, wherein the signal is a change in one of the two wavelengths.

157. The method of either claim 155 or claim 156, wherein the signal is a change at both of the wavelengths.

158. The method according to any one of claims 145-157 further comprising administering a second compound or composition according to any one of claims 1-119 or 177-215.

159. The method of claim 158, wherein the second compound or composition has a different signal.

160. The method of either claim 158 or claim 159, wherein the second compound or composition detects a different disease or disorder.

161. A method of imaing a patient comprising administering to the patient a compound or composition according to any one of claims 1-119 or 177-215, exposing the patient to a light source, and measuring the resultant signal.

162. The method of claim 161, wherein the signal is a change in fluorescence.

163. The method of either claim 161 or claim 162, wherein the signal is change in the adsorbance of visible or untraviolet light.

164. The method according to any one of claims 161-163, wherein the signal decreases in intensity.

165. The method according to any one of claims 161-163, wherein the signal is a change in the λₘₐₓ.

166. The method according to any one of claims 161-165, wherein the patient is imaged in vivo.

167. The mthod according to any one of claims 161-166, wherein the compound targets a microorganism or cellular structure.

168. The method of claim 167, wherein the microorganism is a bacterium.

169. The method of claim 167, wherein the microorganism is a virus.

170. The method according to any one of claims 161-166, wherein the compound targets a cellular structure.

171. The method according to any one of claims 161-170, wherein the compound exhibits absorbance or fluorescence at two or more wavelengths.

172. The method of claim 171, wherein the signal is a change in one of the two wavelengths.

173. The method of either claim 171 or claim 173, wherein the signal is a change at both of the wavelengths.

174. The method according to any one of claims 161-173 further comprising administering a second compound or composition according to any one of claims 1-119 or 177-215.

175. The method of claim 174, wherein the second compound or composition has a different signal.

176. The method of either claim 174 or claim 175, wherein the second compound or composition targets a different microorganism or cellular structure.

177. A compound of the formula: 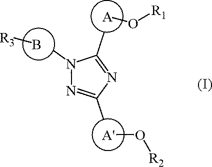
wherein:
A and A′ are arenediyl_{(C≤12)}, heteroarenediyl(_{C≤12}), or a substituted version thereof;
B is —X₁—Y₁—, wherein:
X₁ is arenediyl(_{C≤12}) or substituted arenediyl_{(C≤12)}; and
Y₁ is —C(O)—, —C(O)O—, or -C(O)NR₄-;
R₄ is hydrogen, alkyl_{(C≤6)}, substituted alkyl_{(C≤6)}, or a monovalent amine protecting group;
R₁ and R₂ are each indepdently hydrogen, alkyl_{(C≤12)}, or substituted alkyl_{(C≤12)}; and
R₃ is hydrogen or alkyl_{(C≤12)}, aryl_{(C12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
provided that R₃ is not hydrogen, when Y₁ is —C(O)O—;
or a pharmaceutically acceptable salt thereof.

178. The compound of claim 177 further defined as: 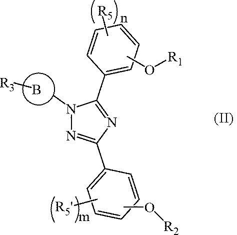
wherein:
B is —X₁—Y₁—, wherein:
X₁ is arenediyl(_{C≤12}) or substituted arenediyl_{(C≤12)}; and
Y₁ is —C(O)—, —C(O)O—, or -C(O)NR₄-;
R₄ is hydrogen, alkyl_{(C≤6)}, substituted alkyl_{(C≤6)}, or a monovalent amine protecting group;
R₁ and R₂ are each indepdently hydrogen, alkyl_{(C≤12)}, or substituted alkyl_{(C≤12)}; and
R₃ is hydrogen or alkyl_{(C≤12)}, aryl_{(C12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
R₅ and R₅′ are each independently hydrogen, halo, or hydroxy; and
m and n are each 1, 2, or 3;
or a pharmaceutically acceptable salt thereof.

179. The compound according to either claim 177 or claim 178 further defined as: 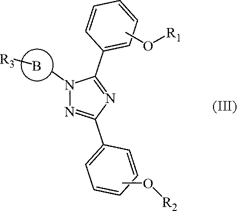
wherein:
B is —X₁—Y₁—, wherein:
X₁ is arenediyl(_{C≤12}) or substituted arenediyl_{(C≤12)}; and
Y₁ is —C(O)—, —C(O)O—, or -C(O)NR₄-;
R₄ is hydrogen, alkyl_{(C≤6)}, substituted alkyl_{(C≤6)}, or a monovalent amine protecting group;
R₁ and R₂ are each indepdently hydrogen, alkyl_{(C≤12)}, or substituted alkyl_{(C≤12)}; and
R₃ is hydrogen or alkyl_{(C≤12)}, aryl_{(C12)}, heteroaryl_{(C≤12)}, heterocycloalkyl_{(C≤12)}, aralkyl_{(C≤12)}, heterocycloalkalkyl_{(C≤12)}, alkoxy_{(C≤12)}, alkylamino_{(C≤12)}, dialkylamino_{(C≤12)}, or a substituted version thereof;
or a pharmaceutically acceptable salt thereof.

180. The compound according to any one of claims 177-179 further defined as: 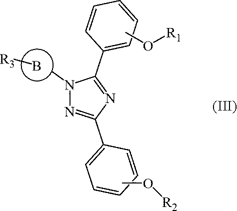
wherein:
B is —X₁—Y₁—, wherein:
X₁ is arenediyl(_{C≤12}) or substituted arenediyl_{(C≤12)}; and
Y₁ is -C(O)NR₄-;
R₄ is hydrogen, alkyl_{(C≤6)}, substituted alkyl_{(C≤6)}, or a monovalent amine protecting group;
R₁ and R₂ are each indepdently hydrogen, alkyl_{(C≤12)}, or substituted alkyl_{(C≤12)}; and or a pharmaceutically acceptable salt thereof.

181. The compound of claim 177, wherein A is arenediyl_{(C≤12)} or substituted arenediyl_{(C≤12)}.

182. The compound of claim 181, wherein A is arenediyl_{(C≤12)}.

183. The compound of claim 182, wherein A is benzenediyl.

184. The compound according to any one of claim 177 and 181-183, wherein A′ is arenediyl_{(C≤12)} or substituted arenediyl_{(C≤12)}.

185. The compound of claim 184, wherein A′ is arenediyl_{(C≤12)}.

186. The compound of claim 182, wherein A′ is benzenediyl.

187. The compound according to any one of 1-10, wherein X₁ is a covalent bond.

188. The compound according to any one of claims 177-186, wherein X₁ is arenediyl_{(C≤12)}.

189. The compound of claim 188, wherein X₁ is benzenediyl.

190. The compound according to any one of claims 177-189, wherein Y₁ is —C(O)—.

191. The compound according to any one of claims 177-189, wherein Y₁ is —C(O)O—.

192. The compound according to any one of claims 177-189, wherein Y₁ is -C(O)NR₄-.

193. The compound of claim 192, wherein R₄ is hydrogen.

194. The compound of claim 192, wherein R₄ is alkyl_{(C≤6)} or substituted alkyl_{(C≤6)}.

195. The compound of claim 194, wherein R₄ is alkyl_{(C≤6)}.

196. The compound of claim 195, wherein R₄ is methyl.

197. The compound according to any one of claims 177-196, wherein R₃ is hydrogen.

198. The compound according to any one of claims 177-196, wherein R₃ is alkyl_{(C≤12)} or substituted alkyl_{(C≤12)}.

199. The compound of claim 198, wherein R₃ is alkyl_{(C≤12)}.

200. The compound of claim 199, wherein R₃ is methyl or ethyl.

201. The compound of claim 198, wherein R₃ is substituted alkyl_{(C≤12)}.

202. The compound of claim 201, wherein R₃ is 2-aminoethyl, 2-(dimethylamino)ethyl, 2-triphenylphosphiumethyl, or 2-cholylethyl.

203. The compound according to any one of claims 177-196, wherein R₃ is heterocycloalkyl_{(C≤12)} or substituted heterocycloalkyl_{(C≤12)}.

204. The compound of claim 203, wherein R₃ is heterocycloalkyl_{(C≤12)}.

205. The compound of claim 204, wherein R₃ is N-methylpiperazinyl or morpholinyl.

206. The compound according to any one of claims 177-196, wherein R₃ is alkoxy_{(C≤12)} or substituted alkoxy_{(C≤12)}.

207. The compound of claim 206, wherein R₃ is alkoxy_{(C≤12)}.

208. The compound of claim 207, wherein R₃ is methoxy.

209. The compound according to any one of claims 177-196, wherein R₃ is alkylamino_{(C≤12)} or substituted alkylamino_{(C≤12)}.

210. The compound of claim 209, wherein R₃ is alkylamino_{(C≤12)}.

211. The compound of claim 210, wherein R₃ is methylamino.

212. The compound according to any one of claims 177-196, wherein R₃ is dialkylamino_{(C≤12)} or substituted dialkylamino_{(C≤12)}.

213. The compound of claim 212, wherein R₃ is dialkylamino_{(C≤12)}.

214. The compound of claim 213, wherein R₃ is dimethylamino.

215. The compound according to any one of claims 177-214 further defind as: 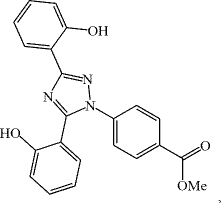 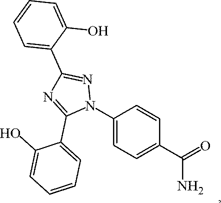 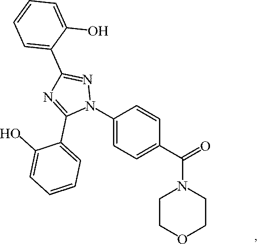 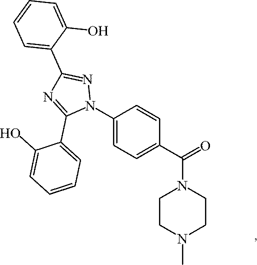 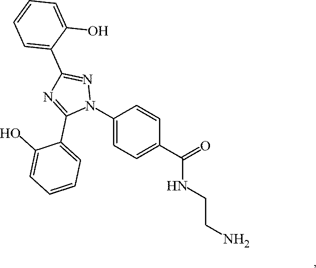 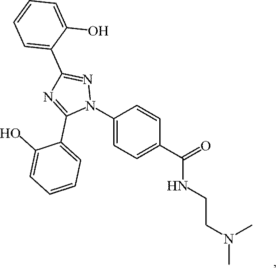 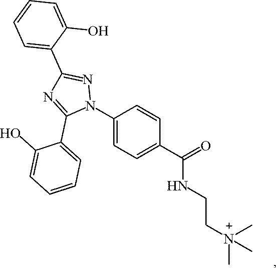 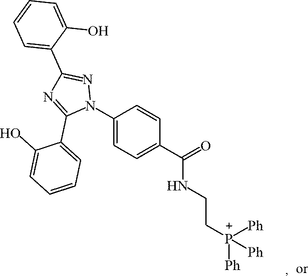 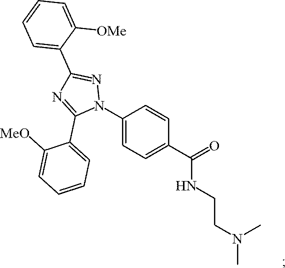
or a pharmaceutically acceptable salt thereof.
